SEQUENCE LISTING (5 sequences)

SEQ ID NO: 1 - DNA, 3054 bp, Saccharomyces cerevisiae
  source 1..3054: mol_type = genomic DNA
atgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggaaagccttaccattcaaatgccaaaggtgatttggcacaaattttacagcttaccagatattttgctgggtccgctgataagtttgacaaaggtgcaaccataccattgacttttaacaagtttgcatatactctaaaagttccttttggcgttgttgctcaaatcgttccatggaattatcctctagctatggcttgttggaaattgcaaggtgccttagcagccggtaacacggttatcatcaaacctgctgagaatacctctctatctctactttattttgctactttaattaaaaaagcaggttttccacctggtgttgtcaatatcgttcctggttatggatcacttgtaggccaagccctagcatctcacatggatatcgacaaaatatcttttacgggaagcaccaaggtcggtggatttgtgttggaagcttccggccaatcgaaccttaaagacgttacactagaatgcggtggtaagtctcctgctctcgtatttgaagatgcagaccttgataaggctatcgattggatagcagctggcattttctacaattcaggacagaattgtaccgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaagggaggaaaagttggacatgttccagacctctgaatttcctattggtggagctaaaggctacttcattcccccaaccatcttcactgatgtcccgcaaacatcgaaactgttacaggatgagatatttggcccggttgtggttgttagcaagttcacaaattatgatgacgctctgaagctggctaatgatacttgctacgggctcgcctctgcggtcttcacaaaagatgtcaagaaagcgcacatgtttgctcgcgatattaaagcaggaactgtttggatcaactcatctaacgatgaagatgttaccgttccttttggcgggtttaaaatgagtggtattggtagagaactggggcaaagtggtgttgatacctatcttcaaacaaaagcaagatatagattatacacatttagaaaattagccaaaagaaaaatgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggtaagcctttccattccaatgctaaacaagacttagcccagattatagaacttacaagatactatgcgggggcggtcgacaagttcaatatgggtgaaaccattccattgacttttaacaagtttgcatatactctaaaagttccttttggcgttgttgctcaaatcgttccatggaattatcctctagctatggcttgtagaaaaatgcaaggtgccttagcggccggtaacacggttatcatcaaacctgctgaaaatacctctctatctctactttattttgctactttaattaaaaaagcaggttttccacctggtgttgtcaatgtcattcctggttatggttccgttgtggggaaagctttaggaacccacatggatatcgacaaaatatcttttacgggaagtactaaggttggcggctcagtattggaagcttccggccaatcgaaccttaaggatatcacactagaatgcggtggtaagtctcctgctcttgtatttgaagatgcagaccttgataaggctatagaatgggtagcaaatggtattttttttaattcgggacagatctgcactgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaaaggaggaaaagttggacatgttccagacctctgaatttcctattggtggagctaaaggctacttcattcccccaaccatcttcactgatgtaccagaaacatctaagttgctgcgtgatgaaatatttggcccggttgtggttgttagcaagttcacaaattatgatgacgctctgaagctggctaatgatacttgctacgggctcgcctctgcggtcttcaccaaagatgtcaagaaagcgcacatgtttgctcgcgatattaaagcaggaactgtttggatcaatcaaaccaatcaagaagaagctaaagttccttttggcggatttaagatgagtggtattggtagagaatcaggcgacaccggcgttgataactatttacaaataaaatcagtccatgtggatctttcattggataaataa

SEQ ID NO: 2 - DNA, 42 bp, Saccharomyces cerevisiae
  source 1..42: mol_type = genomic DNA
agatatagattatacacatttagaaaattagccaaaagaaaa

SEQ ID NO: 3 - DNA, 1521 bp, Saccharomyces cerevisiae
  source 1..1521: mol_type = genomic DNA; strain = ALD2
atgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggaaagccttaccattcaaatgccaaaggtgatttggcacaaattttacagcttaccagatattttgctgggtccgctgataagtttgacaaaggtgcaaccataccattgacttttaacaagtttgcatatactctaaaagttccttttggcgttgttgctcaaatcgttccatggaattatcctctagctatggcttgttggaaattgcaaggtgccttagcagccggtaacacggttatcatcaaacctgctgagaatacctctctatctctactttattttgctactttaattaaaaaagcaggttttccacctggtgttgtcaatatcgttcctggttatggatcacttgtaggccaagccctagcatctcacatggatatcgacaaaatatcttttacgggaagcaccaaggtcggtggatttgtgttggaagcttccggccaatcgaaccttaaagacgttacactagaatgcggtggtaagtctcctgctctcgtatttgaagatgcagaccttgataaggctatcgattggatagcagctggcattttctacaattcaggacagaattgtaccgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaagggaggaaaagttggacatgttccagacctctgaatttcctattggtggagctaaaggctacttcattcccccaaccatcttcactgatgtcccgcaaacatcgaaactgttacaggatgagatatttggcccggttgtggttgttagcaagttcacaaattatgatgacgctctgaagctggctaatgatacttgctacgggctcgcctctgcggtcttcacaaaagatgtcaagaaagcgcacatgtttgctcgcgatattaaagcaggaactgtttggatcaactcatctaacgatgaagatgttaccgttccttttggcgggtttaaaatgagtggtattggtagagaactggggcaaagtggtgttgatacctatcttcaaacaaaagcagttcacataaatctctctttggacaactaa

SEQ ID NO: 4 - DNA, 1521 bp, Saccharomyces cerevisiae
  source 1..1521: mol_type = genomic DNA
atgcctaccttgtatactgatatcgaaatcccacaattgaaaatctctttaaagcaaccgctagggttgtttatcaacaatgagttttgtccatcatcagatggaaagaccatcgaaactgtgaacccagctactggcgaaccgataacatccttccaagcagctaacgaaaaggatgtagacaaagctgtgaaagctgccagggctgcttttgataacgtttggtcgaagacatcttctgagcaacgtggtatttatctttcaaacttattaaaacttattgaggaggagcaagacacacttgccgcattagagactttagacgctggtaagcctttccattccaatgctaaacaagacttagcccagattatagaacttacaagatactatgcgggggcggtcgacaagttcaatatgggtgaaaccattccattgacttttaacaagtttgcatatactctaaaagttccttttggcgttgttgctcaaatcgttccatggaattatcctctagctatggcttgtagaaaaatgcaaggtgccttagcggccggtaacacggttatcatcaaacctgctgaaaatacctctctatctctactttattttgctactttaattaaaaaagcaggttttccacctggtgttgtcaatgtcattcctggttatggttccgttgtggggaaagctttaggaacccacatggatatcgacaaaatatcttttacgggaagtactaaggttggcggctcagtattggaagcttccggccaatcgaaccttaaggatatcacactagaatgcggtggtaagtctcctgctcttgtatttgaagatgcagaccttgataaggctatagaatgggtagcaaatggtattttttttaattcgggacagatctgcactgcaaactcaagagtttatgttcaaagttcgatctacgacaagtttgttgaaaagtttaaagaaactgcaaagaaggagtgggatgttgcaggaaaatttgatccgtttgatgagaaatgcatcgttggtccagttatatcaagtacacagtatgaccgcatcaaaagttacatagaacgtggtaaaaaggaggaaaagttggacatgttccagacctctgaatttcctattggtggagctaaaggctacttcattcccccaaccatcttcactgatgtaccagaaacatctaagttgctgcgtgatgaaatatttggcccggttgtggttgttagcaagttcacaaattatgatgacgctctgaagctggctaatgatacttgctacgggctcgcctctgcggtcttcaccaaagatgtcaagaaagcgcacatgtttgctcgcgatattaaagcaggaactgtttggatcaatcaaaccaatcaagaagaagctaaagttccttttggcggatttaagatgagtggtattggtagagaatcaggcgacaccggcgttgataactatttacaaataaaatcagtccatgtggatctttcattggataaataa

SEQ ID NO: 5 - DNA, 829 bp, Saccharomyces cerevisiae
  source 1..829: mol_type = genomic DNA
ggatctttccgtagggtgaacctggcggagagggatcattaaagaaatttaataattttgaaaatggatttttttgttttggcaagagcatgagagcttttactgggcaagaagacaagagatggagagtccagccgggcctgcgcttaagtgcgcggtcttgctaggcttgtaagtttctttcttgctattccaaacggtgagagatttctgtgcttttgttataggacaattaaaaccgtttcaatacaacacactgtggagttttcatatctttgcaactttttctttgggcattcgagcaatcggggcccagaggtaacaaacacaaacaattttatctattcattaaatttttgtcaaaaacaagaattttcgtaactggaaattttaaaatattaaaaactttcaacaacggatctcttggttctcgcatcgatgaagaacgcagcgaaatgcgatacgtaatgtgaattgcagaattccgtgaatcatcgaatctttgaacgcacattgcgccccttggtattccagggggcatgcctgtttgagcgtcatttccttctcaaacattctgtttggtagtgagtgatactctttggagttaacttgaaattgctggccttttcattggatgttttttttccaaagagaggtttctctgcgtgcttgaggtataatgcaagtacggtcgttttaggttttaccaactgcggctaatctttttttatactgagcgtattggaacgttatcgataagaagagagcgtctaggcgaacaatgttcttaaagtttgacctcaaatccaggtaggagtccccaacgccc

(19) United States
(12) Patent Application Publication
(10) Pub. No.: US 2024/0180985 A1
(43) Pub. Date: Jun. 6, 2024

(54) FOOD AND PHARMACEUTICAL COMPOSITION FOR TREATMENT OF FATTY LIVER AND INFLAMMATION BY RELIEVING ENDOPLASMIC RETICULUM STRESS

(71) Applicant: PICOENTECH Co., LTD., Seongnam-Si (KR)

(72) Inventor: Hung Taeck KWON, Seoul (KR)

(73) Assignee: PICOENTECH Co., LTD., Seongnam-Si (KR)

(21) Appl. No.: 18/519,482

(22) Filed: Nov. 27, 2023

(30) Foreign Application Priority Data
Dec. 1, 2022 (KR) 10-2022-0165785
Nov. 7, 2023 (KR) 10-2023-0152965

(51) Int. Cl. A61K 36/064; A61K 9/00; A61P 1/16

(52) U.S. Cl. CPC A61K 36/064; A61K 9/0053; A61P 1/16

(57) ABSTRACT

Food and pharmaceutical compositions that alleviate endoplasmic reticulum stress and thereby suppress or prevent various diseases caused by endoplasmic reticulum stress. is the embodiments are related to food and pharmaceutical compositions for the prevention or treatment of fatty liver symptoms or hepatitis, containing a lysate of any one or a mixture thereof selected from Saccharomyces cerevisiae KCTC13925BP, KCTC14122BP, KCTC14123BP, KCTC14983BP, KCTC14984BP, and KCTC14985BP.

### REFERENCE TO A SEQUENCE LISTING

In accordance with 37 CFR § 1.831, the present specification makes reference to a Sequence Listing submitted electronically as an .xml file named “PKPA2201KRPR1USA.xml”. The .xml file was generated on Jan. 12, 2024, and is 13000 bytes in size. The entire contents of the Sequence Listing are hereby incorporated by reference.

### TECHNICAL FIELD

The present invention relates to food and pharmaceutical compositions that alleviate endoplasmic reticulum stress and thereby suppress or prevent various diseases caused by endoplasmic reticulum stress. The present invention is directed to food and pharmaceutical compositions that inhibit or prevent fatty liver or hepatitis.

More specifically, the present invention relates to a food or pharmaceutical composition derived from mutated yeast that suppress or prevent liver inflammation, alcoholic fatty liver disease (AFLD), and non-alcoholic fatty liver disease (NAFLD) by alleviating endoplasmic reticulum stress.

The present invention is directed to food and pharmaceutical composition for the prevention or treatment of fatty liver symptoms or hepatitis, containing a lysate of any one or a mixture thereof selected from the group consisting of Saccharomyces cerevisiae KCTC13925BP, KCTC14122BP, KCTC14123BP, KCTC14983BP, KCTC14984BP, and KCTC14985BP.

### BACKGROUND

Causes of fatty liver disease include drinking alcohol and obesity. Fatty liver symptoms may also appear in patients with hyperlipidemia or diabetes who have high blood lipid levels. Fatty liver symptoms may be caused by an increase in toxic aldehydes caused by oxidative stress, as well as drugs that cause endoplasmic reticulum stress, corticosteroids, and female hormones.

When alcohol is consumed, fat synthesis is promoted due to an increase in endoplasmic reticulum stress (ER Stress) in the liver, which causes fat to accumulate in the liver tissue and prevents normal energy metabolism.

Non-alcoholic fatty liver symptoms may occur due to factors other than alcohol consumption. A variety of drugs are being developed to relieve fatty liver symptoms, but to date, no drug has been clearly shown to be effective in treating fatty liver.

Steatohepatitis refers to a case of inflammation accompanied by lipid accumulation in liver tissue and necrosis of liver cells due to insulin resistance and obesity. Steatohepatitis may develop into chronic hepatitis and liver cirrhosis.

The endoplasmic reticulum (ER) is a membrane structure present in the cytoplasm of eukaryotic cells and is connected to the nuclear membrane. The space inside the endoplasmic reticulum occupies 10% of the cell volume and more than 50% of the total cell membrane.

There are two types of endoplasmic reticulum: the rough endoplasmic reticulum (rER), which has a rough shape because many ribosomes that synthesize proteins are attached to the surface, and the smooth endoplasmic reticulum (sER), which has a smooth surface because no ribosomes are attached.

The smooth endoplasmic reticulum (sER) is where membrane lipids and steroid hormones are synthesized, detoxification occurs, and lipids, phospholipids, and steroids are synthesized. In the smooth endoplasmic reticulum, glucose-6-phosphate is dephosphorylated and separated into glucose for excretion, and it also plays a role in detoxifying organic substances, including alcohol, by oxidizing them. Smooth endoplasmic reticulum is abundant in cells that produce liver, muscle cells, steroids, and fat.

Ribosomes are attached to the surface of the rough endoplasmic reticulum (rER) and are responsible for mRNA translation during protein synthesis. In the membrane-enclosed lumen of the endoplasmic reticulum, folding and protein maturation and modification processes that create protein secondary structures take place. Chaperone proteins and enzymes that ripen and modify proteins are distributed in the rough endoplasmic reticulum.

Among proteins synthesized by ribosomes, proteins with an ER signal peptide at the N terminus enter the endoplasmic reticulum. At this time, lipid-soluble membrane proteins stay in the endoplasmic reticulum membrane, and water-soluble proteins enter the endoplasmic reticulum lumen.

Among proteins synthesized by ribosomes, unfolded proteins (UPs) that remain untransformed into the protein's unique secondary structure accumulate in the endoplasmic reticulum, resulting in an increase in endoplasmic reticulum stress (ER Stress). Additionally, in case that abnormally folded proteins (MPs) are not removed and accumulate within the endoplasmic reticulum, ER stress increases.

The response phenomenon that restores or decomposes and removes unfolded and abnormal proteins that cause endoplasmic reticulum stress is called the unfolded protein response (UPR).

Cells respond by activating the unfolded protein response to alleviate endoplasmic reticulum stress or maintain endoplasmic reticulum homeostasis. Proteins involved in the activation of this unfolded protein response include Grp78, IRE-1α, ATF6, and PERK.

Despite alleviating endoplasmic reticulum stress through unfolded protein response, when homeostasis is not maintained smoothly, cells enter the apoptosis pathway. Cell death due to increased ER stress or destruction of endoplasmic reticulum homeostasis may cause metabolic diseases such as viral infectious diseases, obesity, diabetes mellitus, neurodegenerative diseases including dementia, etc., and cause fatty liver, cirrhosis, and cancer.

Reactive oxygen species (ROS) generated during mitochondrial energy production and metabolism process produce toxic substances such as nonenal (HNE), malondialdehyde (MDA), acetaldehyde within cells.

Through the secondary metabolism of these substances, malondialdehyde-acetaldehyde adduct (MAA) and malondialdehyde-lysine adducts (M-lys) are produced.

Through this chain reaction, various modified proteins accumulate in the body, resulting in further increase in oxidative stress.

This increase in oxidative stress affects the energy metabolism process in mitochondria, increasing aldehyde substances such as methylglyoxal and advanced glycation end products (AGEs) in cells. As a result, the disruption of cellular energy metabolism becomes even worse.

In this way, reactive aldehydes such as HNE and MDA, which are the result of lipid peroxidation due to increased oxygen and oxidative stress, and aldehyde substances such as Glyceraldehyde-3-phosphate, an intermediate in glycolysis, accumulate excessively in cells. Accumulation of these substances causes cytotoxicity.

Accumulation of reactive oxygen or reactive aldehydes in cells weaken cellular antioxidant defense systems such as glutathione, ultimately cause an increase in endoplasmic reticulum stress (ER stress) through disruption of energy metabolism and accumulation of unfolded proteins.

When stress occurs in the endoplasmic reticulum of hepatocytes, the smooth endoplasmic reticulum is activated and fat accumulates in the liver, causing fatty liver symptoms and developing into steatohepatitis.

There is an urgent need for the development of food or pharmaceutical compositions that suppress endoplasmic reticulum stress and fat accumulation in the liver, and thereby prevent acute liver damage and steatohepatitis.

### SUMMARY

Despite these many previous studies, to date, a food composition or pharmaceutical composition that suppresses or treats fatty liver or steatohepatitis by suppressing stress applied to the endoplasmic reticulum of liver cells has not yet been developed.

The basic object of the present invention is to provide a mutant yeast-originated composition that blocks the occurrence of endoplasmic reticulum stress by eliminating factors causing endoplasmic reticulum stress in advance, that contains an aldehyde dehydrogenase enzyme decomposing quickly endogenous aldehydes which cause endoplasmic reticulum stress.

Another object of the present invention is to provide a food composition and pharmaceutical composition containing mutant yeast that reduces the possibility of endoplasmic reticulum stress and inhibits or prevents the occurrence of fatty liver or steatohepatitis.

Still yet another object of the present invention is to provide a food composition and a pharmaceutical composition for the prevention and treatment of fatty liver, containing anyone selected from the group consisting of Saccharomyces Cerevisiae KCTC13925BP, KCTC14122BP, KCTC14123BP, KCTC14983BP, KCTC14984BP, KCTC14985BP or a mixture thereof.

The primary object of the present invention as described above can be accomplished by providing a composition originated from mutant yeast containing aldehyde dehydrogenase that can rapidly decompose endogenous aldehydes in advance before occurrence endoplasmic reticulum stress.

Another object of the present invention can also be achieved by providing a food composition or a pharmaceutical composition containing lysate of any one or a mixture thereof (hereinafter abbreviated as KARC) selected from the group consisting of Saccharomyces Cerevisiae KCTC13925BP, KCTC14122BP, KCTC14123BP, KCTC14983BP, KCTC14984BP or KCTC14985BP.

24 and 48 hours after administration of KARC of the present invention to animals with fatty liver symptoms due to endoplasmic reticulum stress in hepatocytes, alleviation of fatty liver symptoms and liver inflammation was observed.

It was confirmed that endoplasmic reticulum stress was reduced in the liver of animals administered KARC of the present invention, fatty liver accumulation was reduced, and expression of liver inflammatory factors was reduced.

### BRIEF DESCRIPTION OF DRAWINGS

Endoplasmic reticulum stress was induced in liver tissue by administering 2 mg/kg Tunicamycin (Tm) to mice.

FIGS. 1 and 2 show the effect of reducing fatty liver in mouse liver tissue 24 and 48 hours after administration of the composition of the present invention.

FIG. 1 shows the liver color of mice 24 hours and 48 hours after administration of tunicamycin and KARC 10 units/kg and 20 units/kg. The liver in which fatty liver disease is induced by tunicamycin becomes lighter in color due to fat. The liver of mice administered KARC returned to red again.

FIG. 2 shows the form of fat in liver tissue through hematoxylin-Eosin (H&E staining). Due to the administration of tunicamycin, the fat in the liver cells increased, but the fat in the liver cells decreased due to the administration of KARC.

FIG. 3 is a graph showing changes in lipid content in liver tissue at intervals of 24 hours and 48 hours after administration of tunicamycin and KARC to mice (n=5).

FIG. 4 is a graph showing changes in lipid content in liver tissue at intervals of 24 hours and 48 hours after administration of tunicamycin and KARC to mice (n=5).

The content of triglyceride (TG) in liver tissue, which was increased by tunicamycin administration, was significantly decreased by KARC administration [FIG. 3]. The content of total cholesterol (T-chol) in liver tissue, which was increased by tunicamycin administration, decreased in a concentration-dependent manner by KARC administration [FIG. 4].

FIGS. 5, 6, 7, 8, and 9 are graphs showing the expression of genes related to endoplasmic reticulum stress (*p<0.05, ** p<0.01) (n=5). Tunicamycin 2 mg/kg and KARC 10 unit/kg and 20 unit/kg were administered to mice, and 24 and 48 hours later, mRNA was isolated from liver tissue. Quantitative real-time reverse transcription polymerase chain reaction (Reverse Transcription quantitative real-time PCR, RT-qPCR) was performed on the isolated mRNA.

FIG. 5 Chop, an endoplasmic reticulum stress marker shown in FIG. 5, represents a C/EBP homologous protein. Endoplasmic reticulum stress changes through regulation of the expression level of proteins involved in inducing apoptosis, such as a decrease in BCL-2 and an increase in BIM due to increased intracellular endoplasmic reticulum stress. The decrease in Chop mRNA expression level by KARC administration shows that endoplasmic reticulum stress is reduced by removal of abnormal proteins in the endoplasmic reticulum that promote cell death [FIG. 5].

FIG. 6 Grp78 represents glucose-regulated protein 78. This is one of the representative chaperone proteins and functions to maintain and regulate endoplasmic reticulum homeostasis. The decrease in the mRNA expression level of Grp78 due to KARC administration means that the accumulation of unfolded proteins is reduced by regulating ATF6 and IRE-1α, and as a result, endoplasmic reticulum stress was alleviated. [FIG. 6].

IRE-1α represents inositol-requiring enzyme 1 alpha. This is a representative unfolded protein response (UPR) indicator. IRE-1α induces abnormally produced and accumulated unfolded proteins to be reformed into normal structures through the secretion of chaperone proteins or removes proteins that will enter the endoplasmic reticulum at the mRNA stage (RIDD, regulated IRE-1α dependent decay). In addition, IRE-1α activates JNK and NF-κB proteins to decompose unfolded proteins by increasing autophagy or inducing apoptosis, thus alleviating endoplasmic reticulum stress and maintaining homeostasis.

FIG. 7 The decrease in the mRNA expression level of IRE-1α due to KARC administration means that the unfolded protein that acts as a cytotoxin is reduced and endoplasmic reticulum stress was alleviated [FIG. 7].

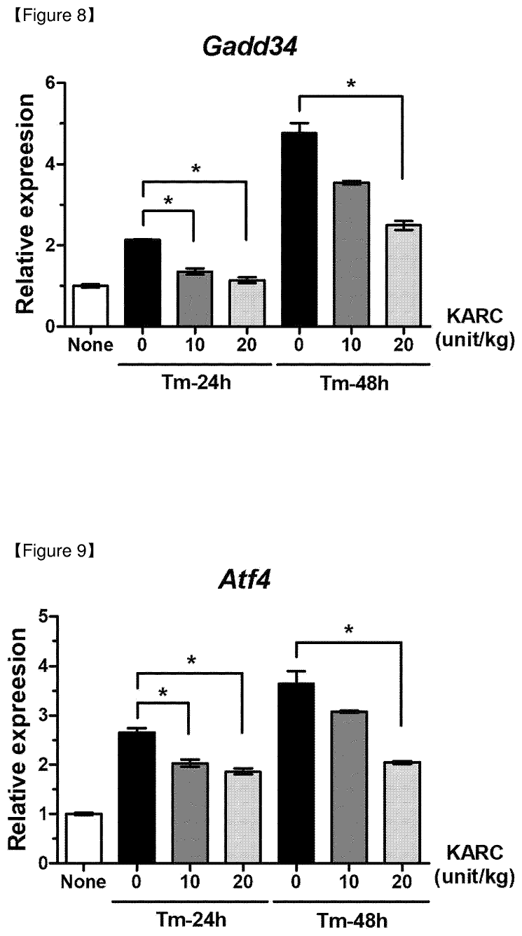

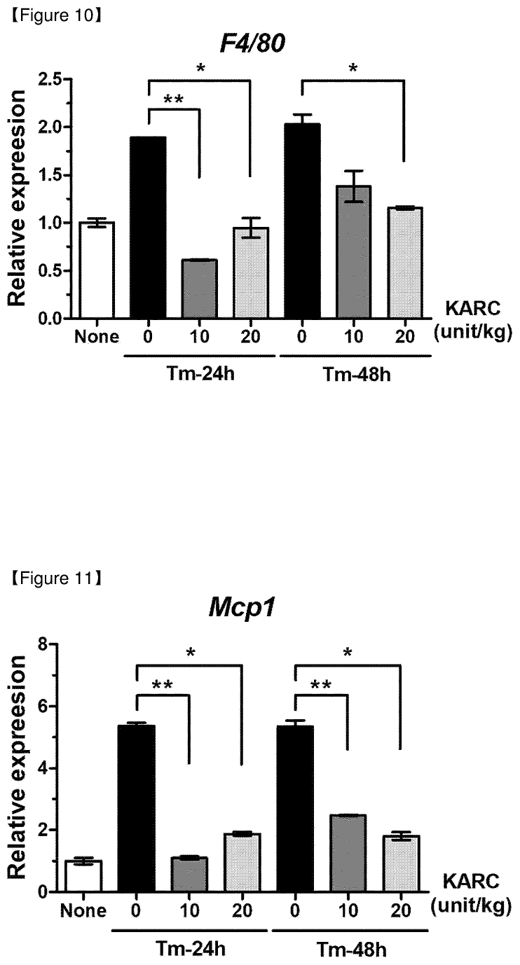

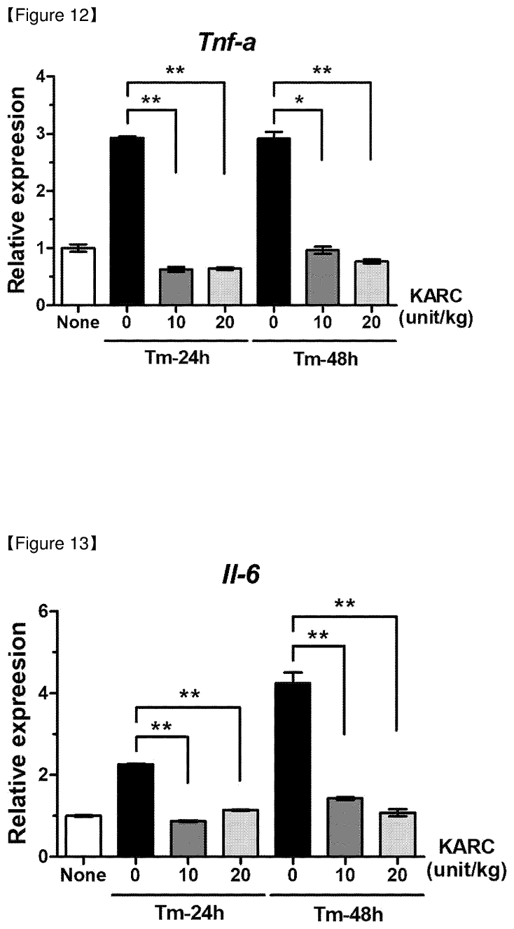

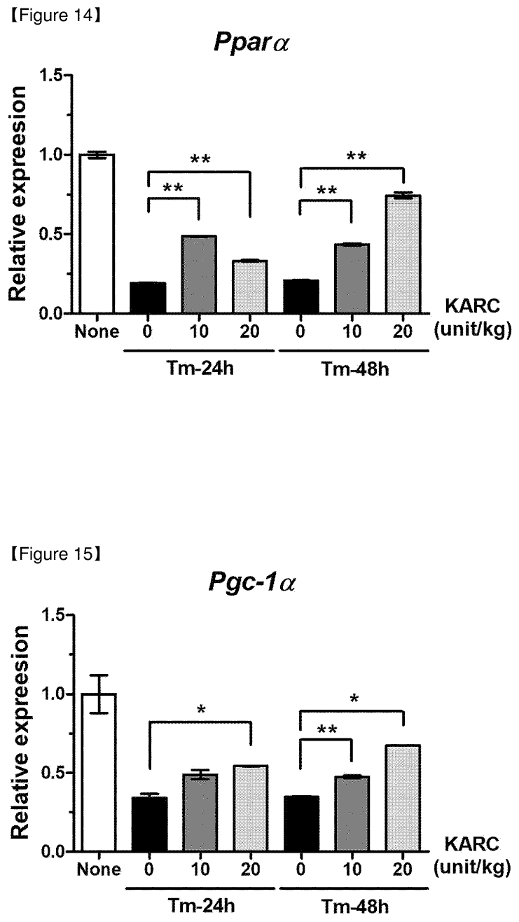

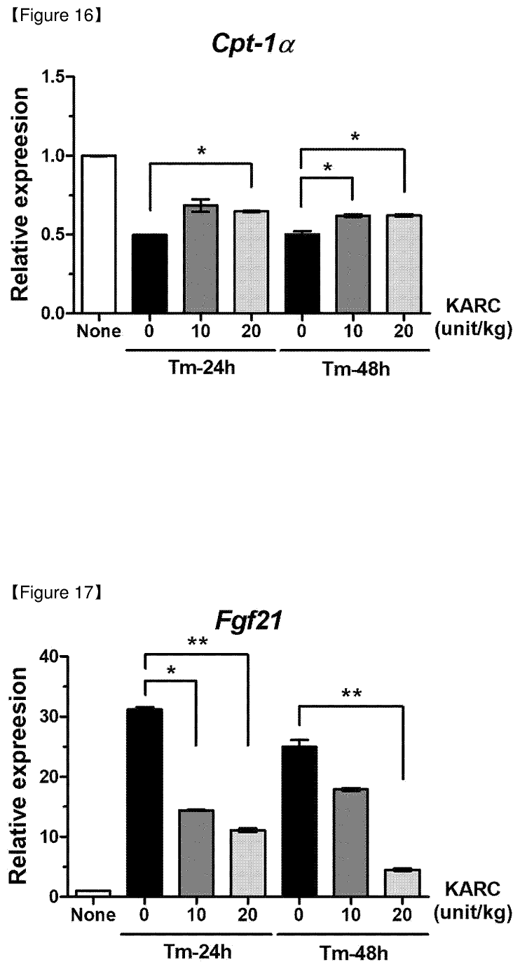

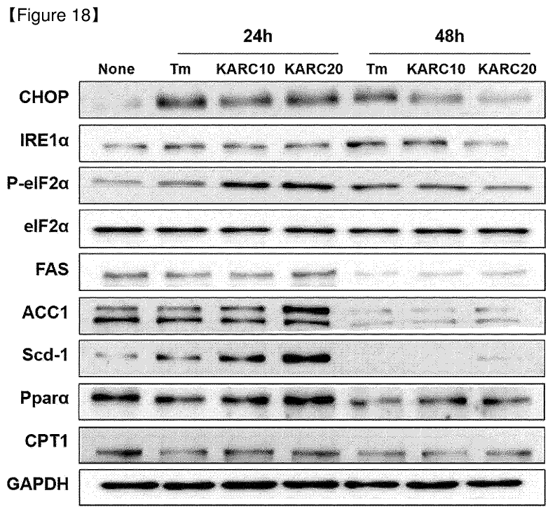

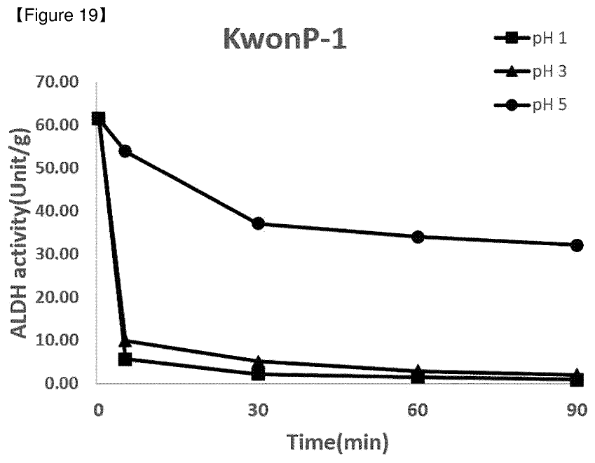

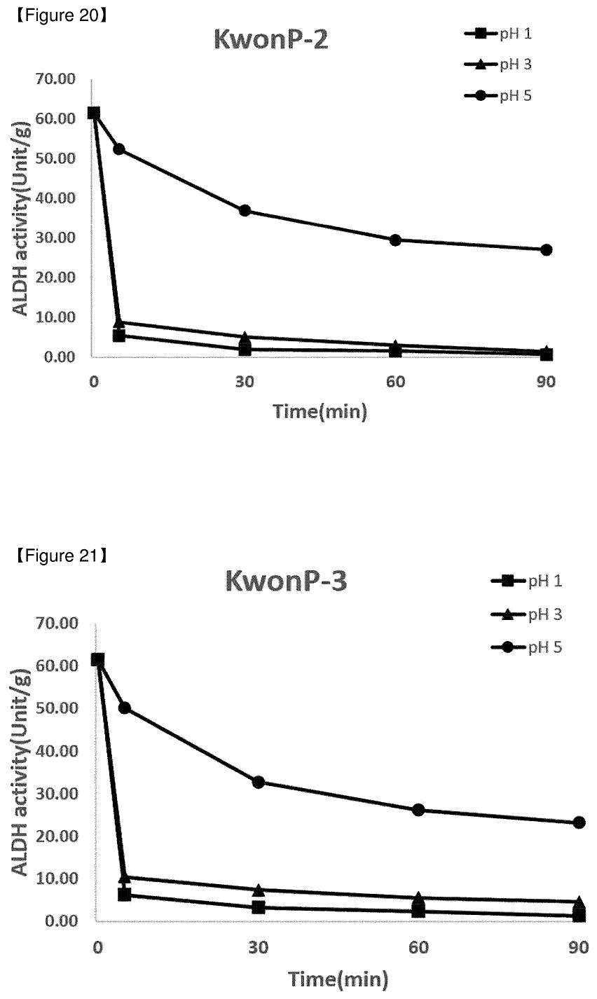

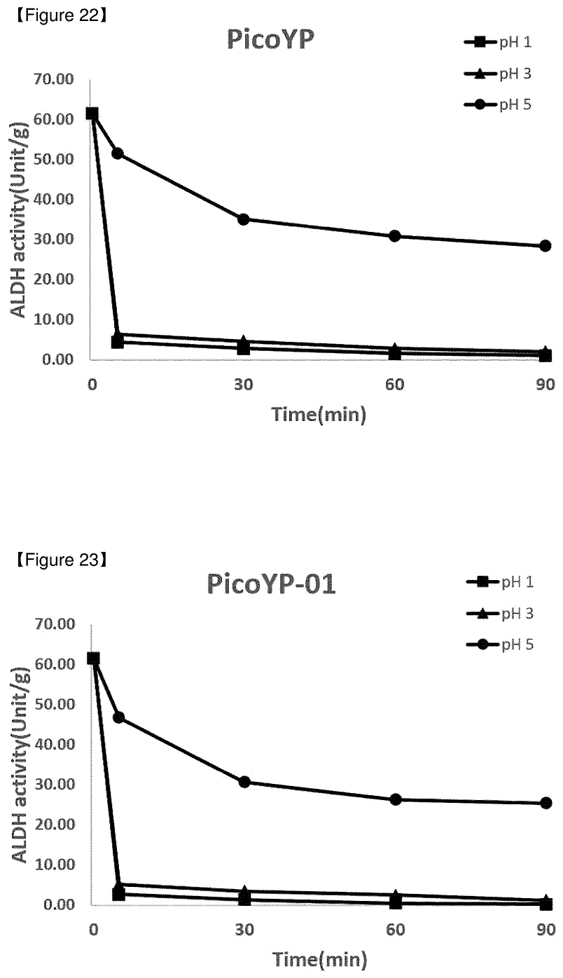

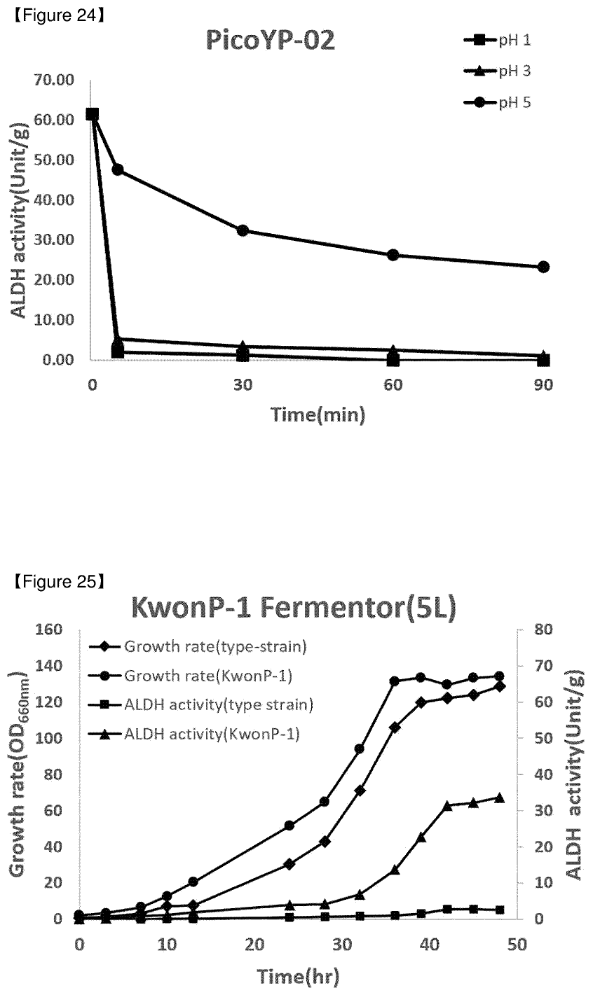

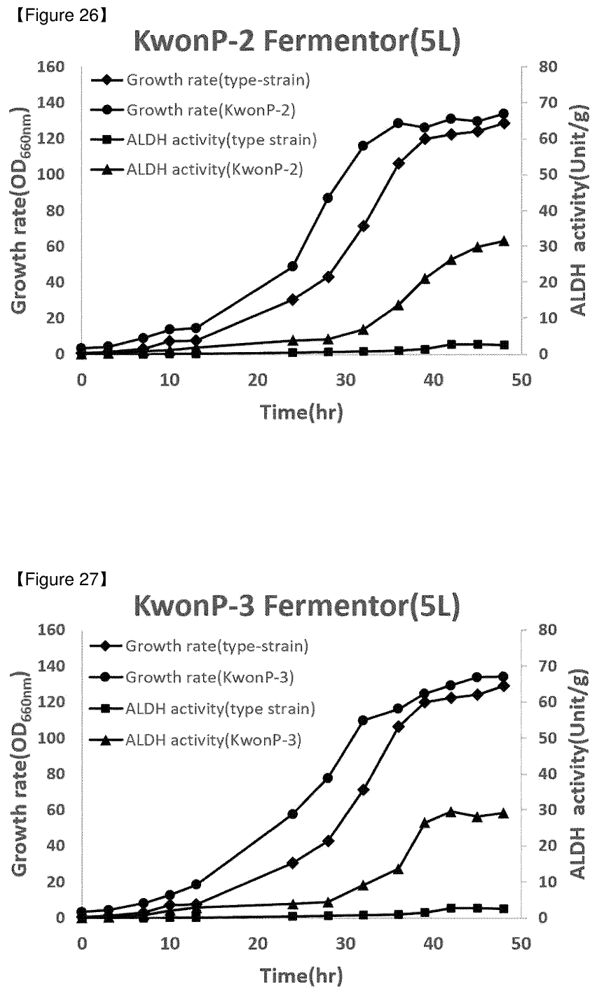

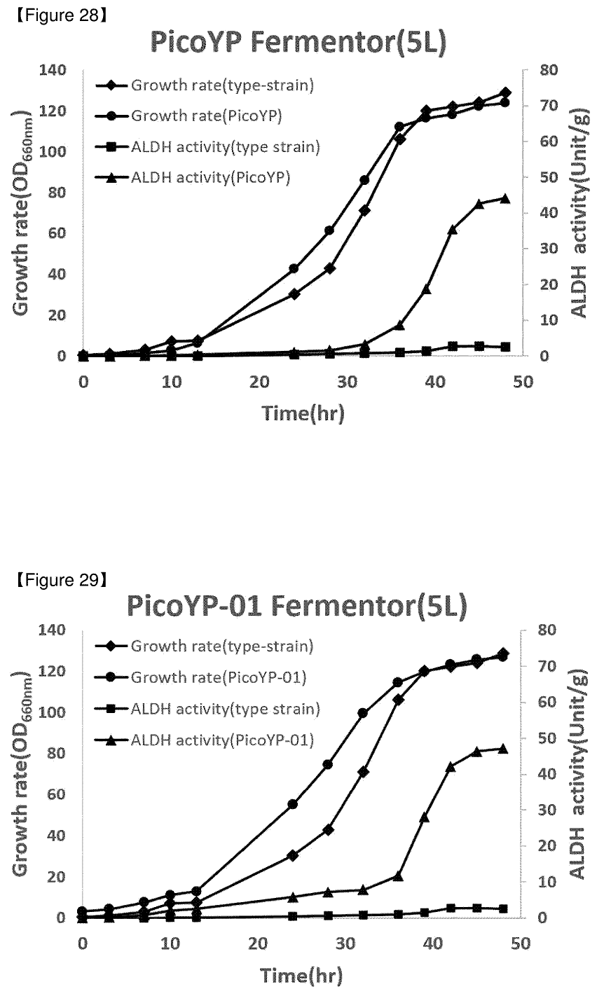

Gadd34 in [FIG. 8] represents a growth arrest and DNA damage inducible protein (growth arrest and DNA damage inducible 34). Gadd34 is a protein that induces eLF2α dephosphorylation in conjunction with the transcription factor ATF4. Dephosphorylation of eLF2α occurs and apoptosis is promoted. Gadd34 acts to remove unfolded proteins by promoting apoptosis due to lack of dephosphorylation of eLF2α or by promoting translation of abnormal proteins.

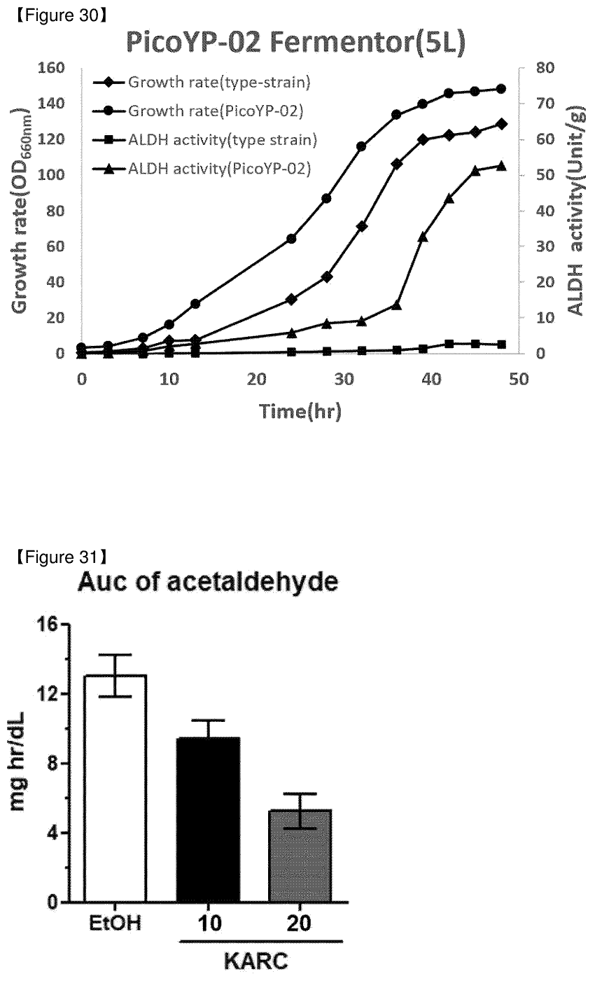

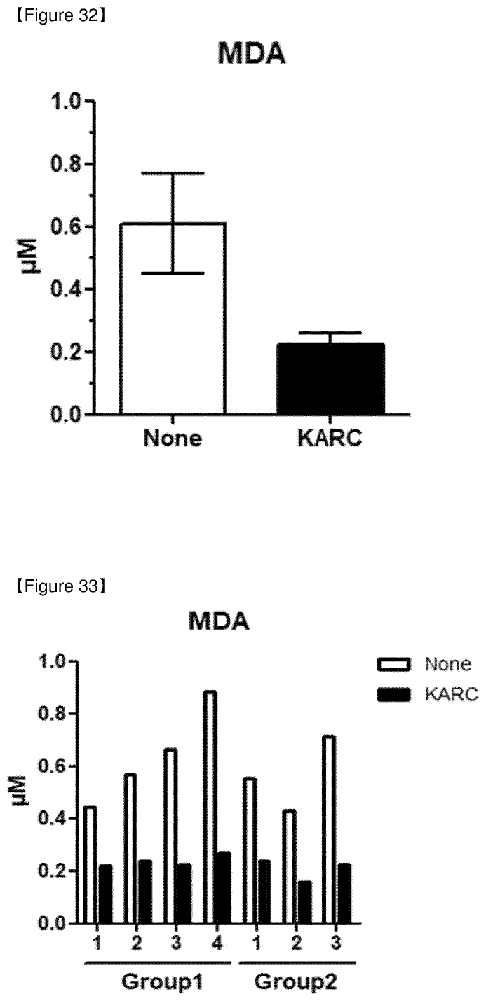

FIG. 9 shows ATF4. ATF4 stands for activating transcriptional factor 4. The decrease in ATF4 expression level shows that endoplasmic reticulum stress is alleviated. The decrease in the mRNA expression level of ATF4 or Gadd34 due to KARC administration in FIGS. 8 and 9 shows that endoplasmic reticulum stress was reduced and the number of dying cells was reduced.

After administering 2 mg/kg tunicamycin to mice, 10 units/kg and 20 units/kg of KARC were administered, and 24 and 48 hours later, mRNA was isolated from the liver tissue of the mice. [FIGS. 10, 11, 12, and 13] show the expression of inflammatory genes through RT-qPCR of isolated mRNA (*p<0.05, ** p<0.01) (n=5).

F4/80 in [FIG. 10] is a unique marker for macrophages. It is present in macrophages that perform phagocytosis and acts to express inflammatory genes. The decrease in the mRNA expression level of F4/80 due to KARC administration indicates that cellular inflammation was reduced.

Mcp1 in [FIG. 11] represents monocyte chemoattracted protein-1. This is a type of chemokine and is expressed in various cells. Mcp1 is an inflammation-linked substance that allows T-cells, monocytes, etc. to gather around inflammation and move easily.

The decrease in Mcp-1 mRNA expression level by KARC administration indicates a decrease in inflammation and inflammatory response.

TNF-α in [FIG. 12] represents tumor necrosis factor-α (tumor necrosis factor-α), a type of cytokine that appears due to increased inflammation and inflammatory response. This shows a phenomenon that increases with increased phagocytosis. The decrease in TNF-α mRNA expression level by KARC administration means that inflammatory substances are reduced, or the inflammatory response is weakened.

Il-6 in [FIG. 13] represents interLeukin-6, which causes inflammation by expressing inflammatory genes. The decrease in the mRNA expression level of Il-6 due to KARC administration means that inflammation is reduced.

After administering 2 mg/kg of tunicamycin to mice, 10 units/kg and 20 units/kg of KARC were administered, and 24 and 48 hours later, mRNA was isolated from liver tissue. RT-qPCR of the isolated mRNA was performed. The expression of genes related to fatty acid oxidation is shown in FIGS. 14, 15, 16, and 17 (*p<0.05, ** p<0.01) (n=5).

Ppar-α in [FIG. 14] represents peroxisome proliferator-activated receptor alpha. It is involved in maintaining homeostasis of lipid metabolism by improving fatty acid oxidation (beta-oxidation) in muscles and liver. The mRNA expression level of Ppar-α was increased by KARC administration. Fat metabolism improved, low-density triglycerides reduced and high-density lipoprotein cholesterol (HDL-Cholesterol, high density lipoprotein cholesterol) is increased. This shows that the liver's lipid metabolism regulation function is restored by KARC administration.

Pgc-1α in [FIG. 15] represents peroxisome proliferator-activated receptor gamma coactivator 1-alpha. This is a protein involved in intracellular mitochondrial biogenesis and hepatic gluconeogenesis. The increase in Pgc-1α mRNA expression level by KARC administration shows that the amount of energy production and energy metabolism in the liver increased.

Cpt-1α in [FIG. 16] represents carnitine palmitoyl transferase 1 alpha. It is in the mitochondrial lipid membrane and is an essential protein involved in fatty acid oxidation (beta-oxidation). This is a protein that converts the acyl group in the fatty acid chain into acylcarnitine, allowing long chain fatty acids to pass through the mitochondrial membrane. The increase in the mRNA expression level of Cpt-1α due to KARC administration shows that energy production and metabolism through fatty acid oxidation (beta-oxidation) in the liver are improved.

Fgf21 in [FIG. 17] represents Fibroblast growth factor 21. It is a fibroblast growth factor that is synthesized in excess in the liver, pancreas, and adipose tissue. The expression level of Fgf21 is regulated by Ppar-γ (peroxisome proliferator-activated receptor gamma) and Ppar-α (eroxisome proliferator-activated receptor alpha). When stress increases, it is activated to adapt to or defend against stress. The decrease in the mRNA expression level of Fgf21 by KARC administration shows that the stress state of hepatocytes was reduced.

After administering 2 mg/kg tunicamycin to the mouse model, 10 unit/kg and 20 unit/kg of KARC were administered. After 24 and 48 hours, western blot was performed on proteins isolated from liver tissue.

FIG. 18 shows the expression of proteins related to endoplasmic reticulum stress, lipid metabolism, and fatty acid oxidation. The expression levels of CHOP, IRE-1α, p-eIF2α, eIF2α, FAS, ACC1, Scd-1, PPARα, and CPT1 proteins were measured (n=5).

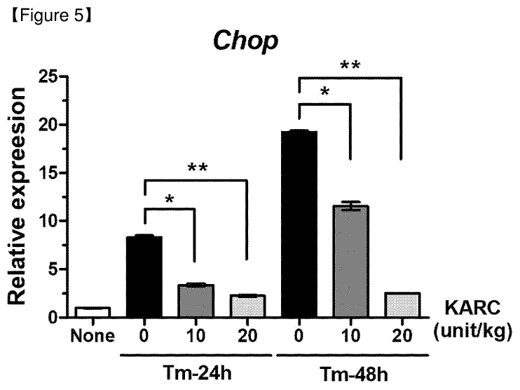

CHOP and IRE-1α, the endoplasmic reticulum stress markers shown in FIG. 18, were increased by tunicamycin administration, as shown in the gene expression results [FIGS. 5 and 7]. In the group administered KARC 20 units/kg, protein expression was decreased, showing that endoplasmic reticulum stress was reduced.

p-eIF2α in [FIG. 18] represents phosphorylation of eukaryotic initiation factor-2α. eIF2α is a gene that is activated when one or more of the four protein kinases (PERK, PKR, GCN2, and HRI) are phosphorylated. It regulates protein synthesis and affects memory ability also.

When cells undergo endoplasmic reticulum stress due to accumulation of unfolded proteins (UP) or misfolded proteins (MP) in the endoplasmic reticulum due to heat, ultraviolet rays, external infections, etc., the phosphatase of p-eIF2α is activated.

Overexpression of this eIF2 protein reduces the total amount of protein, thereby reducing intracellular endoplasmic reticulum stress. The decrease in eIF2 following KARC administration shows that endoplasmic reticulum stress was reduced.

FAS in [FIG. 18] is fatty acid synthase. This is an essential enzyme system involved in lipid biosynthesis (de novo lipogenesis), which is necessary for energy required for cell growth and cell signaling. FAS protein decreased by endoplasmic reticulum stress means that energy metabolism by fatty acid oxidation is reduced. The increase in FAS protein due to KARC administration indicates that energy metabolism was restored by oxidation of fatty acids.

ACC1 in [FIG. 18] is acetyl-CoA carboxylase 1 (ACC1, acetyl-CoA Carboxylase 1). This is an enzyme that produces a metabolite called malonyl-coenzyme A, which is involved in the formation of fatty acids and the burning of fat. The expression of ACC1 is regulated by AMPK (AMP-activated protein kinase), which regulates carbohydrate and fat energy metabolism.

The protein expression of ACC1, which was reduced due to endoplasmic reticulum stress, was restored by KARC administration. This shows that the energy conversion ability through fat oxidation in the liver has been improved.

Scd-1 in [FIG. 18] represents stearoyl-CoA desaturase-1 (Scd-1, stearoyl-CoA desaturase-1). Scd-1, an enzyme mainly present in the endoplasmic reticulum of the liver, is an enzyme that converts saturated fatty acids such as stearic acid, CoA, into unsaturated fatty acids, which are one of the main components of the cell lipid membrane, such as oleic acid. Its expression is regulated by AMPK (AMP-activated protein kinase). The decreased Scd-1 protein expression due to endoplasmic reticulum stress was restored by KARC administration. This shows that lipid metabolism control was improved, and liver cells were recovered. The expression of PPARα and CTP1, which are fatty acid oxidation markers in FIG. 18, is reduced by tunicamycin, as explained in [FIGS. 14 and 16]. The expression of these proteins increased by KARC administration. This shows that energy production and metabolism through oxidation of fatty acids in the liver were improved.

In FIG. 18, GAPDH represents ‘glyceraldehyde-3-phosphate dehydrogenase (GAPDH)’. It is used as a housekeeping gene because it is stably expressed in cells and its expression level does not change easily depending on cell conditions. This was used as a reference marker for protein quantification and shows that the same amount of protein is used.

FIG. 19 shows changes in enzyme activity when KwonP-1 strain was orally administered.

FIG. 20 shows changes in enzyme activity when KwonP-2 strain is orally administered.

FIG. 21 shows changes in enzyme activity when KwonP-3 strain was orally administered.

FIG. 22 shows changes in enzyme activity when the PicoYP strain is orally administered.

FIG. 23 shows changes in enzyme activity when the PicoYP-01 strain is orally administered.

FIG. 24 shows changes in enzyme activity when the PicoYP-02 strain is orally administered.

In FIGS. 19, 20, 21, 22, 23, 24, Kwon P-1, KwonP-2, KwonP-3, PicoYP, Pico YP-01, and PicoYP-02 were orally administered under conditions (1<pH<5) similar to the digestive process of the stomach in human for 90 minutes. The change in ALDH enzyme activity were measured. ALDH enzyme activity was maintained at a minimum of 37.29 unit/g and a maximum of 52.24% at pH=5 (similar to condition observed during food intake). It was confirmed that the enzyme activity was maintained when KARC was administered orally.

FIG. 25 shows the growth curve and enzyme activity when the KwonP-1 strain was cultured in a 5 L fermenter.

FIG. 26 shows the growth curve and enzyme activity when KwonP-2 strain was cultured in a 5 L fermenter.

FIG. 27 shows the growth curve and enzyme activity when KwonP-3 strain was cultured in a 5 L fermenter.

FIG. 28 shows the growth curve and enzyme activity when the Pico YP strain was cultured in a 5 L fermenter.

FIG. 29 shows the growth curve and enzyme activity when the PicoYP-01 strain was cultured in a 5 L fermenter.

FIG. 30 shows the growth curve and enzyme activity when the PicoYP-02 strain was cultured in a 5 L fermenter.

In FIGS. 25, 26, 27, 28, 29, 30, novel mutant strains: KwonP-1, KwonP-2, KwonP-3, PicoYP, PicoYP-01, and PicoYP-01 were respectively cultured using YPD medium in a 5 L fermenter under the same conditions. It was carried out at 30° C. and 200 rpm for 48 hours. When comparing the growth curve (OD660 nm) and ALDH enzyme activity of each strain with that of the type strain, ALDH enzyme activity was at least 10.5 times and up to 18.75 times higher. Pico YP-01 had the highest ALDH activity at 52.68 unit/g, and KwonP-3 had the lowest at 29.5 unit/g.

FIG. 31 is a graph showing the decomposition of acetaldehyde in the human body by KARC.

FIG. 32 is a graph showing the decomposition of malondialdehyde in the human body by KARC.

FIG. 33 is a graph showing stabilization of malondialdehyde in the human body by KARC.

In a test for confirming the reduction of endogenous blood acetaldehyde in the human body [FIG. 31] and blood malondialdehyde reduction [FIG. 32], the effect of administration of KARC appear to reduce acetaldehyde and malondialdehyde, which are biomarkers of hangover, fatigue, and cardiovascular disease.

FIG. 33 shows the effect of lowering oxidative stress by lowering malondialdehyde, a biomarker of oxidative stress and active oxygen, by administration of KARC in a state where oxidative stress was increased due to taking medicine, etc.

### DETAILED DESCRIPTION OF THE EMBODIMENTS

Hereinafter, the method for producing dry powder of KARC of the present invention, the lysate of Saccharomyces cerevisiae KCTC13925BP, KCTC14122BP, KCTC14123BP KCTC14983BP, KCTC14984BP, KCTC14985BP, will be described in more detail.

These examples are for illustrative purposes only of compositions that can achieve the purpose of the present invention, and therefore, the scope of the present invention is not limited only to the compositions described in the following examples.

### [Example 1] Screening Wild Yeast Parent Strain to Proceed Mutation

In the present invention, each suspension of makgeolli (traditional Korean wines) was prepared by mixing various types of makgeolli with a 0.9% NaCl solution. The makgeolli suspension was stirred at 200 rpm for 1 hour. The supernatant containing the yeast wild strain was diluted with YPD (yeast extract peptone dextrose broth) medium. The diluted solution was prepared to be 10-6 times the original solution.

The diluted solution was smeared on YPD agar medium. The agar medium was statically cultured at 30° C. under aerobic conditions for one week. Saccharomyces cerevisiae was primary screened based on morphological characteristics of colonies, growth characteristics at YM medium and microscopic observation.

The ALDH activity and glutathione content of screened Saccharomyces cerevisiae were measured. Parent strain was selected based on ALDH activity and glutathione production.

### 1-1: Measurement of Aldehyde Dehydrogenase

Acetaldehyde reacted with Dinitrophenylhydrazine (DNPH) to form acetaldehyde-hydrazone (Ach-DNPH) compound. Ach-DNPH compounds were detected at 360 nm by HPLC equipped with a C18 column. The amount of aldehyde reduced by the decomposition reaction by aldehyde dehydrogenase (ALDH) was quantified through the amount of the detected Ach-DNPH compound.

The enzyme reaction was carried out at 30° C. by adding 10 ul of the yeast lysate to 990 ul reaction mixture [50 mM potassium phosphate buffer (pH 8.0), 1.5 mM acetaldehyde and 3 mM NADP+]. After the enzyme reaction was completed, 50 ul of 10 mM DNPH was added to induce the formation of Ach-DNPH. Ach-DNPH formation proceeded at 22° C. for 1 hour.

Ach-DNPH formation was terminated by addition of 3M sodium acetate (pH 9). The Ach-DNPH compound formed was separated by adding twice volume of acetonitrile. The separated Ach-DNPH compound (in ACN) was analyzed by injection into HPLC.

The concentration of the Ach-DNPH compound was analyzed at a wavelength of 360 nm by setting HPLC under the condition of developing a mobile phase (acetonitrile, water) on a C18 column at a rate of 1 ml/min. The area value of the chromatogram obtained as a result of HPLC was converted using the material standard curve of aldehyde-DNPH (Sigma-Aldrich) to quantify the concentration of the Ach-DNPH compound. The reduced concentration of Ach-DNPH per minute, 1 mM, was calculated as 1 unit of ALDH. The activity of ALDH was standardized as Unit/mg-protein.

### 1-2: Glutathione Measurement

Yeast cells were harvested by centrifuging 1 ml of Saccharomyces cerevisiae culture medium. A suspension was prepared by adding 1 ml of water to the harvested yeast cells. Glutathione was extracted by stirring the suspension at 1,000 rpm at 85° C. for 2 hours. The suspension was centrifuged to remove yeast cells, and the supernatant was filtered through a 0.22 μm filter to obtain a sample containing glutathione.

The concentration of glutathione in the sample was analyzed by HPLC (Shimazu LC-20AD) equipped with a C18 column. The concentration of glutathione was analyzed at a wavelength of 210 nm under conditions in which the mobile phase (2.02 g/L Sodium 1-heptanesulfonate monohydrate, 6.8 g/L Potassium dihydrogen phosphate, pH 3.0, methanol mixture) was developed at a rate of 1 ml/min. The area value of the chromatogram obtained as a result of HPLC was analyzed using the standard curve of glutathione.

ALDH activity and glutathione content were analyzed for 200 different types of yeast obtained from Korean makgeolli. The 10 types of yeast listed in [Table 1] had higher ALDH activity or glutathione production ability than other yeasts.

The ALDH activity of Yeast #97 was 0.10 Unit/mg-protein, the second highest overall. The glutathione content of Yeast #97 was 0.42%, the highest among all. Yeast #97 was selected as the parent strain and a mutation induction procedure was performed.

TABLE 1

| | ALDH activity | Glutathione | |
|---|---|---|---|
| strain | (Unit/mg-protein) | content (%) | Screening |
| Yeast #6 | 0.06 | 0.38 | |
| Yeast #18 | 0.11 | 0.14 | |
| Yeast #22 | 0.08 | 0.38 | |
| Yeast #41 | 0.14 | 0.22 | |
| Yeast #97 | 0.10 | 0.42 | Selected parent strain |
| | | | (Wild-Type) |
| Yeast #109 | 0.10 | 0.36 | |
| Yeast #112 | 0.09 | 0.40 | |
| Yeast #126 | 0.10 | 0.28 | |
| Yeast #168 | 0.11 | 0.38 | |
| Yeast #197 | 0.08 | 0.41 | |

### [Example 2] Identification of the Parent Strain Used in the Mutagenesis Process

Identification was performed to confirm the exact species of the wild-type parent strain (Yeast #97, Wild-type yeast). To ensure sufficient yeast cells for DNA extraction, only colonies of a single yeast were plated on YPD agar medium. DNA was extracted using a Genomic DNA prep kit (HiGene™, BIOFACT Co., Ltd., Daejeon, Korea) according to the manufacturer's instructions.

To amplify rRNA gene on ITS region of the yeast, polymerase chain reaction (PCR) was performed on yeast chromosomal DNA using the ITS5 (forward) and ITS4 (reverse) primers. DNA sequencing of PCR result was analyzed.

The DNA sequence of the parent strain was isolated using the Bioedit program. The reverse strand of the PCR result was converted into a paired base sequence through a reverse completion process.

It was confirmed that the sequence of the forward strand matched the paired sequence of the reverse strand by the Cluster X program. The parent strain which was matching the sequence information confirmed through the above experimental process was identified by using the BLAST database provided by the U.S. National Center for Biotechnology Information (NCBI). As a result of identification, it was found that rRNA in the ITS of the parent strain was 100% identical to that of Saccharomyces cerevisiae.

### [Example 3] Selection of Mutant Strains with Improved Aldehyde Dehydrogenase Production

The mutation induction process for the wild-type Saccharomyces cerevisiae parent strain was conducted according to the method described in U.S. patent application Ser. No. 17/176,365.

To induce mutations in the yeast parent strain, wild yeast strains that produce both ALDH and glutathione were treated with ethyl methane sulfonate (EMS) or nitrosoguanidine (NGD). Yeast strains in which mutations were induced were exposed to various concentrations of methylglyoxal. A mutant strain with excellent adaptability to methylglyoxal was selected. Selected yeast strains were exposed to various concentrations of lysine. A mutant strain with excellent adaptability to lysine was selected. Thirty mutant strains with excellent adaptability to methylglyoxal and lysine were obtained. Each of the 30 yeasts was evaluated through five characteristics: growth curve, ALDH activity, ADH activity, coenzyme content, and glutathione content.

### 3-1: Growth Characteristics

Saccharomyces cerevisiae is a crab tree positive microorganism and produces ethanol simultaneously with growth under aerobic conditions. Cultivating yeast with high yields requires Saccharomyces cerevisiae with high ethanol tolerance.

YPD media with different ethanol concentrations (no ethanol, 5%, 7%, and 10%) were prepared. Culture medium of Saccharomyces cerevisiae(yeast) adjusted to OD=1 at 660 nm was prepared. Each mixture of the prepared YPD medium and yeast culture medium was diluted at a ratio of 99:1. Finally, YPD media containing yeast with four different concentrations of alcohol were prepared. Each YPD medium mixed with yeast was cultured with shaking at 30° C. and 200 rpm. The growth curve of the mutant strain was measured every 3 hours for 48 hours. The growth curve of each mutant strains were evaluated through three characteristics: time (or period) of lag phase, specific growth rate (OD660 nm/hr) of exponential phase, and maximum density (OD660 nm).

The higher concentration of ethanol in YPD medium, the longer the time taken for the lag phase. The maximum density and specific growth rate decreased. As a result of comparing the maximum density of mutant strains at low concentration (ethanol 5%) and high concentration (ethanol 10%), it was found that in the case of nine mutant strains, 50% of growth was even maintained at high concentration compared to growth at low concentration. The growth characteristics of the nine mutant strains that distinguished them from other strains were a short lag phase and a high specific growth rate.

TABLE 2

| # | 5% ethanol | | | 7% ethanol | | | 10% ethanol | | | |
|---|---|---|---|---|---|---|---|---|---|---|
| # | hr | OD_{660 nm}/hr | OD_{660 nm} | hr | OD_{660 nm}/hr | OD_{660 nm} | hr | OD_{660 nm}/hr | OD_{660 nm} | Selection |
| 1 | 9 | 0.6477 | 22.2 | 15 | 0.5210 | 16.5 | 24 | 0.1968 | 4.81 | |
| 2 | 12 | 0.3675 | 12.34 | 24 | 0.1835 | 4.11 | 36 | 0.0140 | 0.212 | |
| 3 | 9 | 0.4285 | 15.8 | 15 | 0.2888 | 9.12 | 24 | 0.0880 | 2.16 | |
| 4 | 15 | 0.9683 | 25.3 | 15 | 0.8815 | 25.3 | 15 | 0.4085 | 12.4 | K-1 |
| 5 | 12 | 0.7368 | 14.12 | 15 | 0.7337 | 12.23 | 21 | 0.2205 | 5.68 | |
| 6 | 12 | 0.2590 | 9 | 15 | 0.1773 | 5.44 | 33 | 0.0353 | 0.448 | |
| 7 | 24 | 0.9664 | 22.3 | 27 | 0.8467 | 16.8 | 30 | 0.2673 | 5.17 | |
| 8 | 6 | 0.7268 | 23 | 9 | 0.6222 | 21.5 | 15 | 0.3778 | 12.11 | K-2 |
| 9 | 6 | 0.8433 | 22.12 | 9 | 0.7484 | 25.34 | 15 | 0.3005 | 9.41 | |
| 10 | 3 | 0.4880 | 14.5 | 18 | 0.2013 | 6.12 | 24 | 0.0808 | 2.14 | |
| 11 | 3 | 0.2766 | 11.15 | 9 | 0.2223 | 8.22 | 24 | 0.0988 | 2.41 | |
| 12 | 6 | 0.7149 | 21.68 | 9 | 0.5969 | 20.52 | 12 | 0.3317 | 11.4 | K-3 |
| 13 | 9 | 0.6106 | 22.4 | 12 | 0.4906 | 16.4 | 15 | 0.1813 | 5.68 | |
| 14 | 12 | 0.6136 | 20.6 | 24 | 0.3060 | 6.85 | 36 | 0.0278 | 0.41 | |
| 15 | 12 | 0.4759 | 15.45 | 18 | 0.1751 | 5.41 | 33 | 0.0707 | 0.896 | |
| 16 | 9 | 0.8533 | 23.8 | 15 | 0.8065 | 20.9 | 21 | 0.4953 | 12.1 | K-4 |
| 17 | 21 | 0.6016 | 14.85 | 24 | 0.3955 | 8.45 | 24 | 0.0547 | 1.26 | |
| 18 | 12 | 0.7766 | 19.25 | 18 | 0.4437 | 12.4 | 24 | 0.1219 | 2.85 | |
| 19 | 3 | 0.5050 | 14.75 | 9 | 0.4463 | 14.6 | 21 | 0.2521 | 6.23 | |
| 20 | 27 | 0.0666 | 1.41 | 36 | 0.0278 | 0.36 | — | — | — | |
| 21 | 3 | 0.6044 | 22.14 | 9 | 0.6051 | 20.64 | 12 | 0.3247 | 11.1 | K-5 |
| 22 | 24 | 0.5798 | 13.4 | 27 | 0.5080 | 10.1 | 30 | 0.1604 | 3.1 | |
| 23 | 15 | 0.6455 | 16.9 | 15 | 0.5877 | 16.9 | 15 | 0.2003 | 6.3 | |
| 24 | 3 | 0.7269 | 20.4 | 6 | 0.6375 | 18.6 | 12 | 0.3522 | 10.5 | K-6 |
| 25 | 9 | 0.4858 | 16.7 | 15 | 0.3908 | 12.4 | 24 | 0.1476 | 3.6 | |
| 26 | 6 | 0.6559 | 17.2 | 9 | 0.5821 | 19.7 | 15 | 0.3177 | 9.6 | K-7 |
| 27 | 9 | 0.2857 | 10.5 | 15 | 0.1925 | 6.1 | 24 | 0.0587 | 1.4 | |
| 28 | 12 | 0.6315 | 12.1 | 15 | 0.6289 | 10.5 | 21 | 0.1890 | 4.9 | |
| 29 | 6 | 0.5451 | 17.3 | 9 | 0.4667 | 16.1 | 15 | 0.2834 | 9.1 | K-8 |
| 30 | 9 | 0.7826 | 21.8 | 9 | 0.6614 | 19.9 | 15 | 0.3267 | 10.6 | K-9 |

### 3-2: Activity of Alcohol Dehydrogenase (ADH) and Aldehyde Dehydrogenase (ALDH)

The activity of alcohol dehydrogenase (ADH) was measured by adding 10 ul of yeast lysate to 990 ul of the reaction mixture with the composition of 50 mM potassium phosphate buffer (pH 8.0), 2 mM NAD+ and 1% ethanol. The activity of aldehyde dehydrogenase (ALDH) was measured by adding 10 ul of yeast lysate to 990 ul of the reaction mixture with the composition of 50 mM potassium phosphate buffer (pH 8.0), 3 mM NAD+ and 1.5 mM acetaldehyde. The enzymatic reaction of ADH and ALDH was carried out at 30° C. for 5 minutes, and the concentration of NAD(P)H produced as a result of the enzyme reaction was measured through absorbance at 340 nm.

The enzyme activities of nine mutant strains (K-1 to K-9) selected in the present invention were measured. The ADH activity of the mutant strain was a minimum of 382.69 units/g and a maximum of 975.29 units/g. The ADH activity of the mutant strain increased at least 5.1 times and up to 13.1 times compared to the type strain (reference yeast, Saccharomyces cerevisiae KCTC7296). The ALDH activity of the mutant strain was a minimum of 15.23 unit/g and a maximum of 72.16 unit/g. The ALDH activity of the mutant strain increased by at least 5.3 and up to 24.9 times compared to the enzyme activity of the type-strain.

Six mutant strains (K-1, 4, 6, 7, 8, and 9) showed similar increase rate of enzyme activity of ADH and ALDH compared to the type strain. The enzyme activity of ALDH in the three mutant strains (K-2, 3, and 5) was 18.3, 23.2 and 24.9 times higher, respectively, compared to the type-strain. The enzyme activity of ADH in the three mutant strains (K-2, 3, and 5) was 9.7, 11.6, and 13.1 times higher respectively, compared to the type-strain. The rate of increase in enzyme activity of ALDH for the three mutant strains (K-2, 3, and 5) was twice as high as that of ADH.

The present inventors named three novel mutant strains (K-2, 3, and 5) adapted to increase aldehyde dehydrogenase (ALDH) activity as PicoYP, PicoYP-01, and PicoYP-02, respectively. The three novel mutant strains were deposited at the Korea Research Institute of Bioscience and Biotechnology's Biological Resources Center and were assigned the deposit numbers of KCTC14983BP, KCTC14984BP, and KCTC14985BP, respectively.

### 3-3: Content of Coenzyme (NAD and NADP)

NADtotal and NADPtotal in lysates extracted from mutant strains were measured with NADH/NAD+ assay kit and NADPH/NADP+ assay kit, respectively. NAD(P) in the sample was converted to NAD(P)H using NAD(P) cycling buffer and NAD(P) cycling enzyme mix. The chromophoric test reaction was induced with NAD(P) developer measured as absorbance at 450 nm. The chromophoric test reaction was measured as absorbance at 450 nm. The absorbance of the samples was plugged into the equation corresponding to the standard curve, and the NAD(P) total was calculated in the yeast lysate.

The coenzyme content of nine mutant strains (K-1 to K-9) selected in the present invention was measured. The NADtotal of the mutant strains had a minimum of 126 nmole/g and a maximum of 195 nmole/g. The NADtotal of the mutant strain increased at least 7.3 times and up to 10.8 times compared to the type-strain. The NADPtotal content of the mutant strain was a minimum of 2.4 nmole/g and a maximum of 5.8 nmole/g. The NADP total content of the mutant strain increased at least 11.4 times and up to 27.6 times compared to the type-strain.

In the six mutant strains (K-1,4,6,7,8,9), the increase rate of NADPtotal was less than twice the increase rate of NADtotal. The NADPtotal content increase rates of the three novel mutant strains (PicoYP, PicoYP-01, and PicoYP-02) were 25.7, 22.9, and 27.6 times, respectively. The NAD total content increase rates of the three novel mutant strains were 10.8, 9.9, and 11.3 times, respectively. The NADPtotal increase rate of the three novel mutant strains was more than twice the NADtotal increase rate.

### 3-4: Content of Glutathione (GSH)

The glutathione content of the nine mutant strains was measured in the same manner as Example 1-2. The glutathione content of the mutant strains ranged from a minimum of 0.85% to a maximum of 1.05%. The glutathione content of the mutant strain increased at least 2.7 times and up to 3.3 times compared to the type strain. In three novel mutant strains (Pico YP, Pico YP-01, PicoYP-02), the increase rate of ALDH activity and coenzyme content were higher compared to others.

The three novel mutant yeasts (PicoYP, PicoYP-01, PicoYP-02) had similar glutathione production abilities to the existing deposited strains (Kwon P-1, Kwon P-2, Kwon P-3). The three novel mutant yeasts had significantly increased ADH and ALDH enzyme activities and coenzyme contents compared to the existing deposited strains.

TABLE 3

| | Enzyme activity | | Coenzyme concentration | | GSH | | |
|---|---|---|---|---|---|---|---|
| Strain | ADH | ALDH | NADₜₒₜₐₗ | NADPₜₒₜₐₗ | (%) | Name | |
| Type-strain | 74.6 | 2.9 | 17.2 | 0.21 | 0.32 | Reference | KCTC7296 |
| | | | | | | yeast | |
| K-1 | 542.26 | 23.11 | 169.8 | 4.1 | 1.00 | KwonP-1 | KCTC13925BP |
| K-2 | 725.11 | 53.1 | 185 | 5.4 | 0.86 | PicoYP | KCTC14983BP |
| K-3 | 866.41 | 67.4 | 171 | 4.8 | 0.85 | PicoYP-01 | KCTC14984BP |
| K-4 | 625.11 | 31.4 | 176 | 5.1 | 0.98 | KwonP-2 | KCTC14122BP |
| K-5 | 975.29 | 72.16 | 195 | 5.8 | 0.89 | PicoYP-02 | KCTC14985BP |
| K-6 | 458.88 | 16.21 | 154 | 3.1 | 1.05 | — | |
| K-7 | 382.69 | 15.23 | 126 | 2.4 | 1.00 | — | |
| K-8 | 422.17 | 16.19 | 142 | 2.9 | 0.99 | — | |
| K-9 | 533.54 | 20.68 | 167 | 3.2 | 1.00 | KwonP-3 | KCTC14123BP |

TABLE 4

| | Enzyme activity | | Coenzyme concentration | | GSH | | |
|---|---|---|---|---|---|---|---|
| Strain | ADH | ALDH | NADₜₒₜₐₗ | NADPₜₒₜₐₗ | (%) | Name | |
| Type-strain | 1 | 1 | 1 | 1 | 1 | Reference | KCTC7296 |
| | | | | | | yeast | |
| K-1 | 7.3 | 8.0 | 9.9 | 19.5 | 3.1 | KwonP-1 | KCTC13925BP |
| K-2 | 9.7 | 18.3 | 10.8 | 25.7 | 2.7 | PicoYP | KCTC14983BP |
| K-3 | 11.6 | 23.2 | 9.9 | 22.9 | 2.7 | PicoYP-01 | KCTC14984BP |
| K-4 | 8.4 | 10.8 | 10.2 | 17.1 | 3.1 | KwonP-2 | KCTC14122BP |
| K-5 | 13.1 | 24.9 | 11.3 | 27.6 | 2.8 | PicoYP-02 | KCTC14985BP |
| K-6 | 6.2 | 5.6 | 9.0 | 14.8 | 3.3 | — | |
| K-7 | 5.1 | 5.3 | 7.3 | 11.4 | 3.1 | — | |
| K-8 | 5.7 | 5.6 | 8.3 | 13.8 | 3.1 | — | |
| K-9 | 7.2 | 7.1 | 9.7 | 15.2 | 3.1 | KwonP-3 | KCTC14123BP |

### [Example 4] Comparison of Carbon Source Preference

It was investigated the carbon source preference for growth of three mutant strains (KwonP-1, KwonP-2, KwonP-3) with high ALDH and glutathione, for which a domestic patent application was filed on Feb. 18, 2020. Various carbon sources used by the reference yeast strain (KCTC7296) for growth were measured. To find the maximum ability of producing ALDH, it was investigated the carbon source preference for growth of three new mutant strains (Pico YP, PicoYP-01, and Pico YP-02).

The characteristic and novelty of carbon source preference of strains was analyzed by API 50 CHL kit (API systems, BIOMERIEUX, SA, France).

Preparing the 15 ml of conical tube included 8 ml of YPD medium. Each of the seven mutant strains was inoculated into the prepared conical tube.

After culturing the inoculated conical tubes at 30° C. and 200 rpm for 24 hours, each of the seven mutant strains was secured and extracted from the stage of exponential growth phase. To eliminate the influence of the carbon source contained in the residual YPD medium, the yeast was washed three times using a centrifuge. A yeast suspension of 2McFarland concentration was prepared using API 50 CHL medium. The prepared yeast suspension was filled into the tube of the strip. The strip onto which the suspension was dispensed was cultured at 30° C. for 24 hours.

API 50 CHL medium used for API testing was purple. When acids were produced through energy metabolism, API 50 CHL medium turns blue, green, and finally yellow. In the end, it was recorded which type of carbon source was used by mutant strains based on the color change as like: Purple x, Blue+, Green++, and Yellow+++.

All of the seven mutant strains tested used 19 kinds of carbon sources for energy production and growth: L-arabinose, ribose, D-xylose, D-galactose, D-glucose, D-fructose, D-mannose, mannitol, N-acetyl-glucosamine, arbutin, salicin, cellobiose, maltose, lactose, melibiose, sucrose, trehalose, raffinose, gentiobiose.

Rhamnose was used by only three mutant strains: KwonP-1, Pico YP-01, Pico YP-02. Sorbitol was used by four mutant strains: KwonP-1, KwonP-3, PicoYP-01, PicoYP-02. α-methyl-D-mannoside was used by four mutant strains: type strain, KwonP-1, KwonP-2, PicoYP-02. Amygdalin was used by six mutant strains: KwonP-1, KwonP-2, KwonP-3, Pico YP, PicoYP-01, PicoYP-02. D-turanose was used by four mutant strains: type-strain, KwonP-1, KwonP-3, PicoYP-02. D-tagatose was used by three mutant strains: type-strain, KwonP-3, PicoYP-3. Gluconate was used only by type-strain.

Mannitol and sorbitol, which correspond to alcoholic carbon sources, had a significant effect on yeast growth. The three types of novel mutant strains differed from the other four types of yeast in the type of sugar used for growth. The use of the preferred alcoholic carbon source was slightly different between the three new mutant strains (PicoYP. Pico YP-01 and Pico YP-02) [Table 5].

TABLE 5

| | Reference | Kwon | Kwon | Kwon | | | |
|---|---|---|---|---|---|---|---|
| | yeast | P-1 | P-2 | P-3 | PicoYP | PicoYP-01 | PicoYP-02 |
| L-Arabinose | ++ | +++ | ++ | +++ | ++ | ++ | +++ |
| Ribose | +++ | +++ | +++ | +++ | +++ | +++ | +++ |
| D-Xylose | + | ++ | + | + | ++ | ++ | + |
| D-Galactose | + | +++ | ++ | ++ | +++ | ++ | ++ |
| D-Glucose | ++ | +++ | +++ | ++ | +++ | ++ | ++ |
| D-Fructose | ++ | +++ | ++ | ++ | +++ | ++ | ++ |
| D-Mannose | ++ | +++ | +++ | ++ | +++ | ++ | ++ |
| Rhamnose | | + | | | | ++ | ++ |
| Mannitol | + | + | + | + | ++ | +++ | +++ |
| Sorbitol | | | + | + | | +++ | +++ |
| α-Methyl-D- | +++ | + | + | | | | +++ |
| Mannoside | | | | | | | |
| N-Acetyl- | +++ | +++ | +++ | +++ | +++ | +++ | +++ |
| Glucosamine | | | | | | | |
| Amygdalin | | + | + | + | ++ | ++ | ++ |
| Arbutin | +++ | +++ | +++ | +++ | +++ | +++ | +++ |
| Salicin | +++ | +++ | +++ | +++ | +++ | +++ | +++ |
| Cellobiose | +++ | +++ | +++ | +++ | +++ | +++ | +++ |
| Maltose | ++ | +++ | +++ | +++ | +++ | +++ | +++ |
| Lactose | ++ | ++ | ++ | ++ | ++ | ++ | ++ |
| Melibiose | ++ | + | +++ | +++ | ++ | ++ | +++ |
| Sucrose | ++ | +++ | ++ | ++ | +++ | +++ | ++ |
| Trehalose | ++ | + | ++ | ++ | ++ | ++ | ++ |
| Raffinose | ++ | + | + | ++ | ++ | ++ | +++ |
| Gentiobiose | ++ | ++ | ++ | ++ | ++ | ++ | ++ |
| D-Turanose | + | + | | + | | | + |
| D-Tagatose | ++ | | | + | | | ++ |
| Gluconate | + | | | | | | |

### [Example 5] Changes in ALDH Activity of Mutant Strains in Gastric Juice

When KARC is administered orally, in order for the enzyme activity to be maintained in the intestine, the enzyme activity must be passed safely without being destroyed by stomach acid, which secretes powerful proteolytic enzymes such as pepsin.

NaOH solution was added to artificial gastric fluid at pH=1.17 to artificially generate two simulated solutions at pH=3 and pH=5, which resemble the human gastric environment during food digestion. 1 g of KARC was added to 7 ml of artificial gastric fluid and 7 ml of two simulated solutions and mixed at 36.5° C. for 5, 30, 60, and 90 min respectively. NaOH solution was added to reaction mixture to adjust acidity to pH=7, respectively. 10 ml of sample for analysis were taken from the adjusted solution at pH=7, respectively. The activity of ALDH was analyzed from each sample.

Under the condition of pH=1.17, the ALDH activity of the sample decreased by more than 92.88% compared to the control group during 5 minutes of reaction. Under the condition of pH=1.17, the ALDH activity of the sample decreased by an average of 98.89% for 90 minutes. The ALDH activity of the samples decreased by an average of 96.66% over 90 min at pH=3 and 56.83% at pH=5. Ultimately the ALDH activity at pH=3 and 5 remained relatively higher than that at pH=1.17 during the 90-min reaction.

In detail, the ALDH activity of KwonP-1 (KCTC13925BP) at pH=1.17 decreased by 90.94% compared to the control group to 5.57 unit/g when reacted for 5 minutes. The ALDH activity of KwonP-1 decreased by 98.57% to 0.88 unit/g for 90 minutes [FIG. 19]. The enzyme activity at pH=3 and pH=5 remained relatively higher than at pH=1.17. When reacted for 90 minutes, the ALDH activity of KwonP-1 decreased by 96.66% to 5.57 units/g at pH=3, and decreased by 98.57% to 0.88 units/g at pH=5.

The ALDH activity of KwonP-2 (KCTC14122BP) at pH=1.17 decreased by 91.18% to 5.43 unit/g when reacted for 5 minutes. The ALDH activity of KwonP-2 decreased by 98.81% to 0.73 unit/g for 90 minutes [FIG. 20]. At pH 3 and pH 5, higher enzyme activity was maintained than at pH 1.17. When reacted for 90 minutes, the ALDH activity decreased by 97.62% to 1.47 units/g at pH=3, and decreased by 56.11% to 26.99 units/g at pH=5.

The ALDH activity of KwonP-3 (KCTC14123BP) at pH=1.17 decreased by 89.99% to 6.16 unit/g when reacted for 5 minutes. The ALDH activity of KwonP-3 decreased by 97.85% to 1.32 unit/g for 90 minutes [FIG. 21]. At pH 3 and pH 5, higher enzyme activity was maintained than at pH 1.17. When reacted for 90 minutes, the ALDH activity decreased by 92.61% to 4.55 units/g at pH=3 and decreased by 62.31% to 22.18 units/g at pH=5.

The ALDH activity of Pico YP (KCTC14983BP) at pH=1.17 decreased by 92.84% to 4.40 unit/g when reacted for 5 minutes. The ALDH activity of PicoYP decreased by 98.33% to 1.03 unit/g for 90 minutes [FIG. 22]. Higher enzyme activity was maintained at pH 3 and pH 5. When reacted for 90 minutes, the ALDH activity decreased by 96.66% to 2.05 units/g at pH=3, and decreased by 53.97% to 28.31 units/g at pH=5.

The ALDH activity of Pico YP-01(KCTC14984BP) at pH=1.17 decreased by 95.71% to 2.64 unit/g when reacted for 5 minutes. The ALDH activity of PicoYP-01 decreased by 99.76% to 0.15 unit/g for 90 minutes [FIG. 23]. At pH 3 and pH 5, higher enzyme activity was maintained than gastric fluid. When reacted for 90 minutes, the ALDH activity decreased by 98.21% to 1.10 units/g at pH=3, and decreased by 58.74% to 25.38 units/g at pH=5.

The ALDH activity of Pico YP-02(KCTC14985BP) at pH=1.17 decreased by 96.66% to 2.05 unit/g when reacted for 5 minutes. The ALDH activity of PicoYP-02 decreased by 99.76% to 0.15 unit/g for 90 minutes [FIG. 24]. At pH 3 and pH 5, higher enzyme activity was maintained than in gastric juice. When reacted for 90 minutes, the ALDH activity decreased by 98.21% to 1.10 units/g at pH=3 and decreased by 62.08% to 23.32 units/g at pH=5.

pH 1.17 is the pH of the raw gastric juice secreted. When you eat food, the pH rises from 3 to 5 when raw gastric fluids and food mix in the stomach, so it is unlikely that a pH of 1.17 will be reached. Nevertheless, ALDH activity in the mutant strain was retained even at pH 1.17, which is an extreme condition.

In the end, the ALDH enzyme activity of the novel mutant strains (Pico YP, Pico YP-01, PicoYP-02) was maintained at 2 unit/g to 5 unit/g even though it decreased from 92% to 97% under strongly acidic conditions of pH=1.17. 2-5 units of enzyme activity remain, which is sufficient to function in the intestines. It even remained higher at pH=3 and pH=5 compared to pH=1.17. This was the reason for reaching the conclusion that new mutant strains (Pico YP, PicoYP-01, PicoYP-02) could be administered orally.

### [Example 6] Growth Characteristics of 5 L Fermenter Cultures

Each was inoculated into YPD medium (2% peptone, 1% yeast extract, 2% glucose) and primary seed culture was performed at 30° C. and 200 rpm for 18 hours. 20 ml of cultured seed was inoculated into 1980 ml of YPD medium and cultured again in 5 L. Cultivation in a 5 L culture tank was carried out at 30° C. and 200 rpm for 48 hours. Growth curve at OD660 nm and enzyme activity were analyzed using 10 ml of sample collected from secondary culture.

The maximum density (OD660 nm) of KwonP-1 (KCTC13925BP) was 134.4. The maximum density of KwonP-1 was 4.35% higher than that of the type-strain (KCTC7296). The growth curve characteristics and specific growth rate (OD660 nm/hr) of KwonP-1 were similar to those of the type-strain. The ALDH activity of KwonP-1 was 33.6 unit/g. The ALDH activity of KwonP-1 was 11.96 times higher than that of the type-strain [FIG. 25].

The maximum density (OD660 nm) of KwonP-2 (KCTC14122BP) was 133.8. The maximum density of KwonP-2 was 3.88% higher than that of the type-strain. The growth of KwonP-2 ended earlier than that of the type-strain. The specific growth rate (OD660 nm/hr) of KwonP-2 was 14.8% higher than that of the type-strain. The ALDH activity of KwonP-2 was 31.5 unit/g. The ALDH activity of KwonP-2 was 11.21 times higher than that of the type-strain [FIG. 26].

The maximum density (OD660 nm) of KwonP-3 (KCTC14123BP) was 134.1. The maximum density of KwonP-3 was 4.12% higher than that of the type-strain. The growth of KwonP-3 ended earlier than that of the type-strain. The specific growth rate (OD660 nm/hr) of KwonP-3 was 6.08% higher than that of the type-strain. The ALDH activity of KwonP-3 was 29.5 unit/g. The ALDH activity of KwonP-3 was 10.5 times higher than that of the type-strain [FIG. 27].

The maximum density (OD660 nm) of PicoYP (KCTC14983BP) was 123.8. The maximum density of PicoYP was 3.88% higher than that of type-strain. The growth curve characteristics of PicoYP were similar to those of type-strain. The specific growth rate (OD660 nm/hr) of PicoYP was 6.22% higher than that of the type-strain. The ALDH activity of PicoYP was 44.2 unit/g. The ALDH activity of PicoYP was 15.73 times higher than that of the type-strain [FIG. 28].

The maximum density (OD660 nm) of PicoYP-01 (KCTC14984BP) was 126.9. The maximum density of PicoYP-01 was 1.47% higher than that of the type-strain. The growth curve characteristics of PicoYP-01 were similar to those of type-strain. The specific growth rate (OD660 nm/hr) of PicoYP-01 was 2.14% higher than that of the type-strain. The ALDH activity of PicoYP-01 was 47.1 unit/g. The ALDH activity of PicoYP-01 was 16.76 times higher than that of the type-strain [FIG. 29].

The maximum density (OD660 nm) of PicoYP-02 (KCTC14985BP) was 148.1. The maximum density of PicoYP-02 was 14.99% higher than that of the type-strain. The growth curve of PicoYP-02 was located at the top compared to the type-strain. The specific growth rate (OD660 nm/hr) of PicoYP-02 was 9.64% lower than that of the type-strain. The ALDH activity of PicoYP-02 was 52.68 unit/g. The ALDH activity of PicoYP-02 was 18.75 times higher than that of the type-strain [FIG. 30].

### [Example 7] Preparation of Mutant Strain Lysates (KARC)

To preserve the enzymes (ALDH, ADH) contained in the mutant enzyme lysate, proteases were removed and inhibited. To preserve the enzymes (ALDH, ADH) contained in the mutant enzyme lysate cell debris was removed. The dried product or lysate of the mutant strain was mixed to prepare the KARC composition.

The mutant strain and the medium in which it was cultured contained various substances, such as yeast metabolites and proteolytic enzymes secreted by yeast. In order to extract and preserve ALDH, coenzyme, and glutathione present in yeast, it is necessary to sufficiently remove substances outside the yeast fungus, and for this purpose, a washing process was performed. Washing of the mutant strain was carried out by dispensing 40 ml of culture medium into 50 ml conical tubes, centrifuging at 13,000 rpm for 15 minutes, and removing the supernatant.

As a result of centrifugation, residual medium remained inside the pellet produced by the yeast bacteria clumping together. After adding 30 ml of purified water, the pellet was sufficiently loosened by vortex, and the previous process was repeated three times to sufficiently remove the remaining medium.

The ethanol resistance of yeast is known to be up to 13%, and yeast bacteria die when exposed to high concentrations of ethanol. The washed pellet was sufficiently dissolved using 10 ml of 20% ethanol solution to induce the death of yeast bacteria. The pellet dissolved in ethanol was stirred at 100 rpm for 30 minutes to proceed with the yeast death process. When the reaction time was completed, 30 ml purified water was added to lower the ethanol concentration to 5%. The previous washing process was repeated three times to sufficiently remove ethanol.

To preserve ALDH and ADH from the decomposition action of proteases present in yeast cells, 10 ml of 1×PBS was prepared by dissolving 2 tablets of protease inhibitor (Pierce protease inhibitor mini tablets, EDTA-free, Thermo Scientific). The above solution was added to the washed yeast pellet and sufficiently released.

To prepare a lysate of the mutant strain prepared in the present invention, 4 g of glass beads were added and stirred to break the yeast cell wall. To prevent denaturation of the enzyme due to the heat generated during the process of crushing the yeast, vortex for 30 seconds and ice incubation for 30 seconds were repeated six times.

After the yeast cell wall disruption was completed, 10 ml of 100 mM potassium phosphate buffer was added and mixed by vortex for 3-5 seconds. It was centrifuged at 13,000 rpm for 15 minutes to remove cell structures such as yeast cell walls and glass beads. The supernatant was filtered through a 0.2 μm filter (Minisart Syringe Filter, Sartorius, Goettingen, Germany) to prepare the KARC composition.

To preserve the enzymes (ALDH, ADH) contained in the mutant enzyme lysate, intracellular proteases were removed and inhibited, and cell debris such as cell walls were removed. The KARC composition was prepared with a lysate selected from the 6 mutant strains (KwonP-1, KwonP-2, KwonP-3, PicoYP, PicoYP-01, PicoYP-02), or a mixture thereof in a free ratio [Table 6].

KARC 1 was manufactured from KwonP-1. The enzyme activity of ADH and ALDH of KARC 1 were 461.4 unit/g and 28.6 unit/g, respectively. In KARC 1, the content of coenzymes of NADtotal and NADPtotal were 176.2 nmole/g and 5.1 nmole/g, respectively. The GSH content of KARC 1 was 0.98 wt %.

KARC 2 was manufactured from KwonP-2. the enzyme activity of ADH and ALDH of KARC 2 were 482.1 unit/g and 29.8 unit/g, respectively. In KARC 2, the content of coenzymes of NADtotal and NADPtotal were 175.4 nmole/g and 5.2 nmole/g, respectively. The GSH content of KARC 2 was 0.96 wt

KARC 3 was manufactured from KwonP-3. the enzyme activity of ADH and ALDH of KARC 2 were 477.5 unit/g and 28.1 unit/g, respectively. In KARC 3, the content of coenzymes of NADtotal and NADPtotal were 177.2 nmole/g and 5.1 nmole/g, respectively. The GSH content of KARC 3 was 1.00 wt %.

KARC 4 was manufactured from PicoYP. the enzyme activity of ADH and ALDH of KARC 2 were 586.8 unit/g and 33.8 unit/g, respectively. In KARC 4, the content of coenzymes of NADtotal and NADPtotal were 184.3 nmole/g and 5.7 nmole/g, respectively. The GSH content of KARC 4 was 0.84 wt %.

KARC 5 was manufactured from PicoYP-01. the enzyme activity of ADH and ALDH of KARC 5 were 621.6 unit/g and 38.2 unit/g, respectively. In KARC 5, the content of coenzymes of NADtotal and NADPtotal were 186.9 nmole/g and 5.6 nmole/g, respectively. The GSH content of KARC 5 was 0.84 wt %.

KARC 6 was manufactured from PicoYP-02. the enzyme activity of ADH and ALDH of KARC 5 were 664.1 unit/g and 41.6 unit/g, respectively. In KARC 6, the content of coenzymes of NADtotal and NADPtotal were 195.0 nmole/g and 5.8 nmole/g, respectively. The GSH content of KARC 6 was 0.88 wt %.

KARC was manufactured by freely mixing dry powders and lysates prepared from six deposit strains. The average enzyme activities of ADH and ALDH in the composition of KARC were 547.6 unit/g and 33.1 unit/g, respectively. The average contents of coenzyme NADtotal and coenzyme NADPtotal in the composition of KARC were 180.4 nmole/g and 5.4 nmole/g, respectively. The average content of glutathione in the composition of KARC was 0.84 wt %.

The aldehyde decomposition ability of KARC was kept on during the lysate production process. KARC showed the ability to remove endogenous aldehydes such as HNE, MDA, and 3,4-dihydroxyphenyl acetaldehyde (DOPAL).

TABLE 6

| | | ADH | ALDH | NADtotal | NADPtotal | GSH |
|---|---|---|---|---|---|---|
| Name | Strain | (Unit/g) | (Unit/g) | (nmole/g) | (nmole/g) | (wt %) |
| KARC1 | KwonP-1 | 461.4 | 28.6 | 176.2 | 5.1 | 0.98 |
| KARC2 | KwonP-2 | 482.1 | 29.8 | 175.4 | 5.2 | 0.96 |
| KARC3 | KwonP-3 | 477.5 | 28.1 | 177.2 | 5.1 | 1.00 |
| KARC4 | PicoYP | 586.8 | 33.8 | 184.3 | 5.7 | 0.84 |
| KARC5 | PicoYP-01 | 621.6 | 38.2 | 186.9 | 5.6 | 0.83 |
| KARC6 | PicoYP-02 | 664.1 | 41.6 | 195.0 | 5.8 | 0.88 |
| KARC | average | 547.6 | 33.1 | 180.4 | 5.4 | 0.91 |

### [Example 8] Analysis of Sequence of ALDH Contained in the Mutant Strain

It was investigated the differences between both ALD (yeast aldehyde dehydrogenase) of the mutant strains and parent strain. Whole genome sequencing was performed on the parent strain and mutant strains of KwonP-1, KwonP-2, KwonP-3, PicoYP, PicoYP-01, and PicoYP-02. The mutant strain cells were obtained by culturing pure strains on solid medium. The genome sequence of the mutant strain obtained were analyzed.

Among ALDs (yeast aldehyde dehydrogenases) in the novel mutant strains, ALD2(SEQ ID NO:3) was found to be condensed with ALD3(SEQ ID NO:4) on chromosome 13. A non-coding region of 689 nucleotides was located between the ALD2 and ALD3 coding genes

The ALD2 and ALD3 existed continuously in the same genome. ALD2 and ALD3 encoded respective aldehyde dehydrogenases. ALD2 coding gene was almost similar to ALD3, consist of 1,521 nucleotides and 506 amino acids, but had an 8.2% difference in sequence. ALD2 and ALD3 they were identified as separate aldehyde dehydrogenases that differed from each other in 125 base sequences (8.2%).

In the six mutant strains (KwonP-1, KwonP-2, KwonP-3, PicoYP, PicoYP-01, PicoYP-02), there is no stop codon at the end of the ALD2 sequence, so proteins are synthesized continuously. As a result, a new, larger ALDH enzyme is created by linking a part of ALD2 and ALD3[SEQ ID NO: 1].

ALD2[SEQ ID NO. 3] of the type-strain (KCTC7296) consisted of 30 nucleotide sequences (5′-GTTCACATAAATCTCTCTTTGGACAACTAA-3′) coding 9 amino acids (N-VHINLSLDN-C) at the terminal, excluding the stop codon.

ALD2 of the six mutant strains consisted of specific 42 nucleotide sequences (5′-AGATATAGATTATACACATTTAGAAAATTAGCCAAAAGAAAA-3′) coding 14 amino acids (N-RYRLYTFRKLAKRK-C) between 5′-terminal of ALD2 and ALD3, [SEQ ID NO. 2].

There was no stop codon at the end of the sequence in ALD2 coding gene by deleted from the 1492^{nd} nucleotide of ALD2 to 647^{th} nucleotide of non-coding region. Finally, the six deposited mutant strains had new mutated gene consist of total 3,054 bases coding novel ALD. [SEQ ID NO: 1].

### [Example 9] the Effectiveness of KARC in Reducing Fat of Liver Induced by Endoplasmic Reticulum (ER Stress)

Administration of tunicamycin (Tm) to animals inhibits N-glycosylation of proteins, resulting in the formation of unfolded proteins (UP). Administration of tunicamycin to animals causes ER Stress. Tunicamycin inhibits the N-acetylglucosamine transferase enzyme in eukaryotic cells. Inhibition of N-acetylglucosamine transferase inhibits the formation of N-acetylglucosamine lipid metabolites and the glycosylation of newly synthesized proteins.

In endoplasmic reticulum (ER), proteins synthesized on the ribosome undergo higher-order structural and post-translational modifications, leading to the folding and assembly of glycoproteins. The inhibition of glycoprotein production by tunicamycin interferes with protein maturation. As a result of disrupted protein maturation, misfolded or unfolded proteins are produced. The misfolded or unfolded proteins fail to assemble correctly and accumulate in ER, causing ER Stress. ER Stress causes cell death.

To investigate the efficacy of KARC in inhibiting endoplasmic reticulum (ER) Stress-mediated acute fatty liver, the present inventor analyzed the effects of KARC and the fat-related morphology of liver tissue in model animals with induced ER Stress.

In detail the C57BL/6J male mice (Jungang Laboratories, Korea), a model animal for ER Stress induction, were breading in facility at 23° C. and 60-70% humidity with a 12-hour (light), 12-hour (dark) light/dark cycle (6 a.m.-6 p.m. light, 6 p.m.-6 a.m. dark). The mice had free access to water in pathogen-free facility.

The liver sample size of the experimental animals was estimated based on analysis. ER Stress was induced by administering 2 mg/kg tunicamycin into the peritoneal cavity of the mice, and the effect of KARC was tested at 24 and 48 hours intervals.

KARC were prepared from dry powder or lysate of Saccharomyces cerevisiae KwonP-1, KwonP-2, KwonP-3, PicoYP, PicoYP-01, or PicoYP-02. The prepared KARC contain glutathione, aldehyde dehydrogenase (ALDH), alcohol dehydrogenase (ADH), and the coenzymes NAD and NADP.

Mice were anesthetized with isoflurane (Gyeonggi Hana Pharmaceutical) and sacrificed at 9 am. To analyze the morphology of the liver tissue, H&E staining was used to confirm its structure. H&E tissue staining was accomplished.

Lipids in liver tissue were extracted using methanol and chloroform according to Folch's method. Total cholesterol levels in liver tissue were quantified using a total cholesterol assay kit (AM 202-K, Asan Pharmaceuticals) and the absorbance of samples and standards was measured at 500 nm.

Triglyceride levels in liver tissue were quantified using a triglyceride assay kit (AM 157S-K, Asan Pharmaceuticals, Seoul, Korea). The quantified triglyceride levels in liver tissue were measured by measuring the absorbance of samples and standards at 550 nm using an Infinite 200 PRO (Tecan Trading AG, Switzerland).

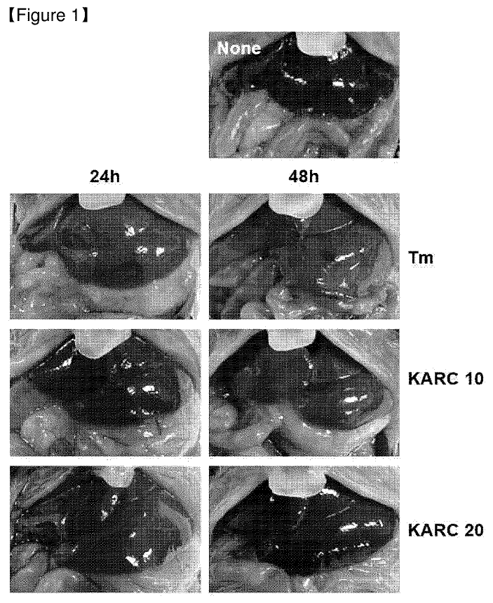

Tunicamycin induced the appearance of fatty liver in the liver tissue [FIG. 1].

In the tunicamycin injected group, the fatty liver phenomenon persisted and remained discolored at 24 and 48 hours after injection. In the groups administered with 10 units/kg and 20 units/kg of KARC, the higher the concentration of KARC, the higher the rate of fatty liver recovery.

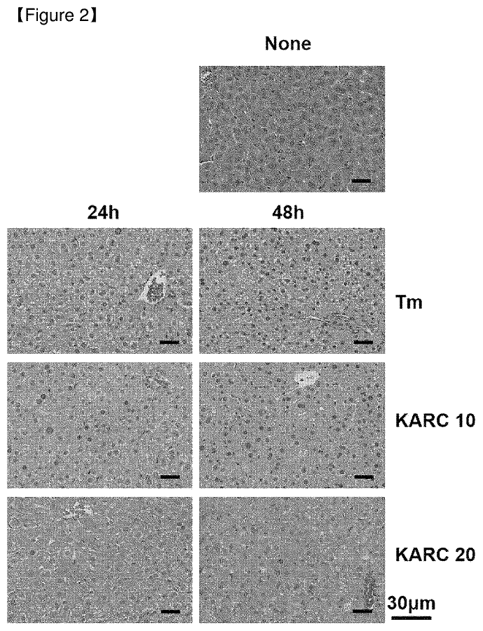

Tissue morphology analysis by H&E staining also showed that the amount of fat in the liver tissue was less in the KARC administered group compared to the vehicle group [FIG. 2].

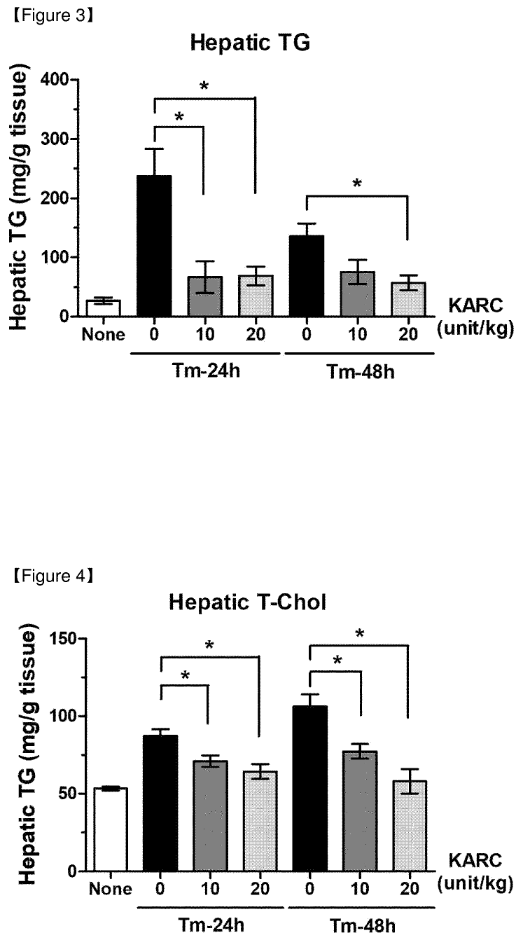

The content of triglycerides [FIG. 3] and total cholesterol [FIG. 4] in liver tissue after induction of antifoam stress was determined. Triglycerides and total cholesterol in liver tissue were increased in the livers of mice with induced ER Stress. Both triglycerides and total cholesterol in liver tissue were reduced in the livers of mice administered with KARC. KARC, a Saccharomyces cerevisiae lysate, was effective in the treatment of fatty liver.

### [Example 10] Changes in ER Stress-Related Factors Following KARC Administration

Tunicamycin prevents the transfer of N-acetylglucosamine 1-phosphate to the dolichol phosphate site of proteins. As a result, tunicamycin inhibits the binding of sugar to NH3 of asparagine, a protein building block.

Tunicamycin suppresses glycosylated protein synthesis by inhibiting entry into the S phase of the cell cycle and inhibits DNA synthesis by extending the G1 phase. ER Stress-induced model animal was prepared by injecting a normal mouse model with tunicamycin. The effect of KARC was investigated by administering 20 units/kg of the mutant yeast lysate KARC to the ER Stress-induced model animals for 24 or 48 hours.

Acute fatty liver-related factors were analyzed in liver tissue from mice as soon as tunicamycin administration or KARC administration.

To investigate an effect of inhibiting acute fatty liver formation, we analyzed in detail factors related to endoplasmic reticulum stress, inflammation, lipid synthesis, and fatty acid oxidation in the liver tissues of mice injected with tunicamycin or KARC.

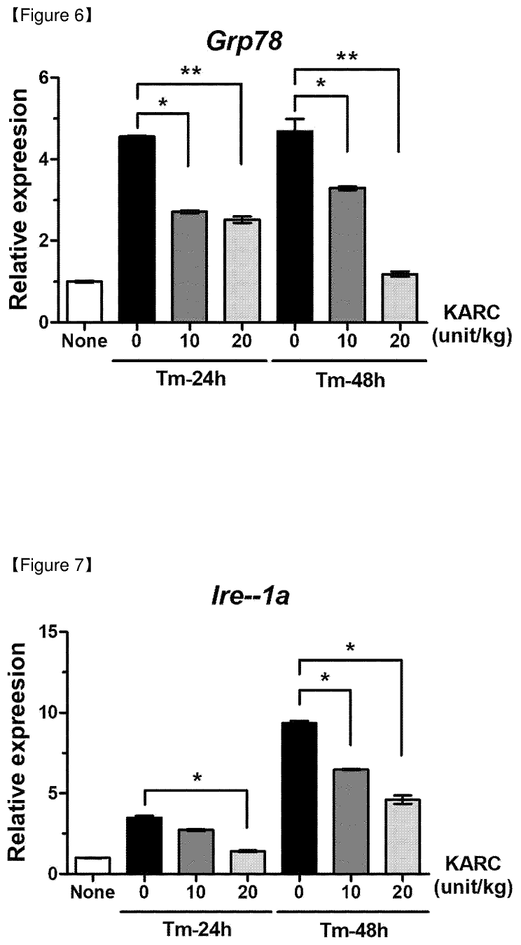

The expression levels of mRNA and protein of ER Stress-related genes Chop [FIG. 5], Grp78 [FIG. 6], Ire-1α [FIG. 7], Gadd34 [FIG. 8], and Atf4 [FIG. 9] were measured in the liver tissues of ER stress-induced mice. Expression levels of inflammatory genes F4/80 [FIG. 10], Mcp1 [FIG. 11], Tnf-α [FIG. 12], and Il-6 [FIG. 13] were measured in liver tissues of ER stress-induced mice. The expression levels of Ppar-α [FIG. 14], Pgc-1α [FIG. 15], Cpt-1α [FIG. 16], and Fgf21[FIG. 17] genes related to fatty acid oxidation, were measured.

Total RNA was prepared from liver tissue of mice with ER Stress induced by the TriZol procedure (Invitrogen). Using total RNA as a template, cDNA was synthesized using the iScript cDNA synthesis kit (Bio-Rad Laboratories, Hercules, CA, USA).

To measure the mRNA expression levels of various genes, qPCR was performed with a CFX96™ real-time PCR system (Bio-Rad Laboratories).

The mRNA levels were normalized to the expression of ribosomal protein L32 by calculating the delta-delta threshold cycle method. The primer sequences of the genes used for qPCR are listed in [Table 7]. Western blot analysis was performed by extracting protein samples from liver tissues of ER stress-induced mice.

Liver tissue lysates (extracts or digests) were extracted using T-PER (Tissue Protein Extraction Reagent; Thermo Scientific, Rockford, IL, USA) supplemented with protease and phosphatase inhibitors (Thermo Scientific). The proteins in lysate of liver tissue were diluted with 5× sample buffer (EBA-1052, ELPIS BIOTECH, Seoul, Korea) and heated at 95° C. for 5 min. Proteins were separated by 5-15% Tris-HCl SDS/PAGE gel electrophoresis and transferred to nitrocellulose membranes (GE Healthcare, Uppsala, Sweden).

All immunoblots were probed by chemiluminescence-enhanced HRP-conjugated secondary antibodies (Clarity Western ECL Substrate, Bio-Rad Laboratories). Protein bands were detected with a chemiluminescence imaging system (Fusion Fx, Vilber Lourmat, Eberhardzell, Germany).

The expression of ER Stress-related genes was analyzed in the liver tissues of KARC administered mice after induction of ER Stress by qPCR. As shown in [FIGS. 5, 6, 7, 8, and 9], all groups administered with KARC improved the expression of ER Stress-related genes whose expression was increased by induction of ER Stress. Mice with induced ER Stress are known to have increased expression of inflammatory genes in liver tissue.

The expression of marker genes related to inflammatory genes was analyzed. As shown in [FIGS. 10, 11, 12, and 13], all genetic markers were reduced in expression by KARC.

The expression of genes related to fatty acid oxidation was analyzed. As shown in [FIGS. 14, 15, 16, and 17], fatty acid oxidation was improved by the reduction of ER Stress in the KARC administered group.

When analyzing the expression of proteins related to ER Stress, CHOP, Ire-1α, and p-eIF2α expression was reduced in all cases of KARC administration. The expression of proteins related to lipid metabolism and fatty acid oxidation was analyzed. In all cases, KARC administration improved ER Stress and reduced the expression of proteins involved in lipid metabolism and fatty acid oxidation [FIG. 18].

TABLE 7

| Name | | Sequence (5′−>3′) |
|---|---|---|
| Chop | F | CAGTCATGGCAGCTGAGTCC |
| | R | TAGGTGCCCCCAATTTCATC |
| Grp78 | F | GAAAGGATGGTTAATGATGCTGAG |
| | R | GTCTTCAATGTCCGCATCCTG |
| Atf4 | F | TGGAAACCATGCCAGATGAG |
| | R | GATGGCCAATTGGGTTCACT |
| Ire-1α | F | GTGGTCTCCTCTCGGGTTC |
| | R | CCGTCCCAGGTAGACACAAAC |
| Gadd34 | F | GACCCCTCCAACTCTCCTTC |
| | R | CTTCCTCAGCCTCAGCATTC |
| F4/80 | F | TGAGATTGTGGAAGCATCCGAG |
| | R | ACAGCAGGAAGGTGGCTATG |
| Mcp1 | F | ACTGAAGCCAGCTCTCTCTTCC |
| | R | TTCCTTCTTGGGGTCAGCACAG |
| Tnf-α | F | CCCTCACACTCAGATCATCTTCT |
| | R | GCTACGACGTGGGCTACAG |
| Il-6 | F | TCTATACCACTTCACAAGTCGGA |
| | R | GAATTGCCATTGCACAACTCTTT |
| Pparα | F | AGAGCCCCATCTGTCCTCTC |
| | R | ACTGGTAGTCTGCAAAACCAAA |
| Pgc-1α | F | AACAAGCACTTCGGTCATCCCTG |
| | R | TTACTGAAGTCGCCATCCCTTAG |
| Cpt-1α | F | CTCCGCCTGAGCCATGAAG |
| | R | CACCAGTGATGATGCCATTCT |
| Fgf21 | F | GTACCTCTACACAGATGACGACCAA |
| | R | CGCCTACCACTGTTCCATCCT |

### [Example 11] Effect of Reducing Oxidative Stress

Reactive oxygen species or oxidative stress increases when drinking alcohol due to excessive acetaldehyde (Ach) produced by alcohol dehydrogenase (ADH). Aldehyde dehydrogenase (ALDH) acts to convert it into acetic acid and excrete it out of the body. In the case of aldehyde dehydrogenase gene mutation or excessive aldehyde caused by excessive alcohol cause peroxidation of fat.

The resulting acetaldehyde and malondialdehyde worsen oxidative stress and interfere with mitochondrial energy metabolism. Endoplasmic reticulum stress is induced through the accumulation of denatured proteins in cells, leading to cell death.

The concentration of blood acetaldehyde was measured over time following alcohol consumption [FIG. 31]. The area under the curve (AUC) of blood acetaldehyde (Ach) was 13.02±1.18 mg·h/dL for alcohol consumption alone. When administered at a dose of KARC 10 units/kg, the area under the curve (AUC) of blood acetaldehyde (Ach) significantly decreased by 26.13% compared to alcohol consumption alone, measuring 9.39±1.07 mg·h/dL (P=0.005).

At a dose of KARC 20 units/kg administration, the AUC of blood acetaldehyde (Ach) decreased significantly by 55.71% compared to alcohol consumption alone, measuring 5.22±0.99 mg·h/dL (P<0.001). When comparing the KARC 10 units/kg administration group with the KARC 20 units/kg administration group, the blood acetaldehyde (Ach) in the KARC 20 units/kg group decreased significantly (P=0.034). KARC demonstrated dose-dependent reduction in the total amount of blood acetaldehyde (Ach) over time.

The reduction in blood acetaldehyde (Ach) concentration due to KARC administration has a positive impact on reducing oxidative stress and promoting health.

The concentration of blood malondialdehyde (MDA) was measured during the chemotherapy period [FIG. 32]. The concentration of blood MDA in the control group was 0.607±0.161 μM. The group undergoing treatment with KARC showed a significant 63.3% reduction in blood MDA concentration, measuring 0.223∓0.033 μM compared to the control group (P<0.001).

In the control group, the blood MDA concentration ranged from 0.427 μM to 0.885 μM with a substantial variability. In the KARC administration group, the range was significantly reduced, with values ranging from 0.158 μM to 0.269 μM. This not only confirmed the effect of reducing blood MDA concentration but also stabilizing it, as demonstrated in [FIG. 33].

Various factors, such as drug intake, stress, and intense physical exercise, lead to an increase in intracellular reactive oxygen species. This triggers lipid peroxidation reactions and oxidative processes in endogenous amines such as dopamine, norepinephrine, serotonin, histamine, and more. Reactive aldehyde compounds, including 4-hydroxynonenal (HNE), malondialdehyde (MDA), acetaldehyde (Ach), and dopamine-induced aldehyde, accumulate within cells, exacerbating oxidative stress.

These aldehydes subsequently react with surrounding proteins and undergo secondary metabolic processes to form stable end products such as Malondialdehyde-acetaldehyde adduct (MAA) and Malondialdehyde lysine adducts (M-lys adducts), known as Advanced Lipid Peroxidation End Products. The accumulation of these products exerts toxic effects on various cells, further intensifying oxidative stress.

This cumulative oxidative stress disrupts mitochondrial energy metabolism within cells and leads to the buildup of aldehyde intermediates in aldehyde-based sugar metabolism, including methylglyoxal (MG) and glyceraldehyde-3-phosphate (GA3P). The chain reaction involving aldehydes results in the accumulation of stable final glycoxidation products known as advanced glycation end products (AGEs), which weaken intracellular antioxidant defense systems like glutathione (GSH). These processes elevate endoplasmic reticulum (ER) stress, leading to increased cellular apoptosis in nerve cells.

The increase in reactive oxygen species and oxidative stress is associated with elevated levels of reactive aldehydes like HNE and MDA, as well as modified proteins such as advanced glycation end products (AGEs) and advanced lipid peroxidation end products (ALEs). This cascade of events is known to involve mutual reinforcement and amplification, leading to heightened endoplasmic reticulum stress (ER stress).

KARC administration effectively regulated malondialdehyde, a marker for active oxygen and oxidative stress, demonstrating the potential for reducing oxidative stress and improving the constancy of endoplasmic reticulum (ER) stress. KARC significantly reduced malondialdehyde concentrations in the bloodstream, illustrating its capability to reduce active oxygen and oxidative stress.

By lowering the levels of acetaldehyde and malondialdehyde in human blood, KARC exhibited its potential to prevent and remedy ER stress through the reduction of active oxygen and oxidative stress.

### [Example 12] Acute Oral Administration Test

### 12-1. Preparation of Experimental Animals

The experimental animals were female and male ICR mice (7 weeks old). The received ICR mice were acclimatized for 7 days. The general symptoms of the adopted mice were observed during the acclimatization period, and only healthy animals were used for short-term administration toxicity tests. Feed and water were consumed ad libitum. Based on the average body weight of about 20 g the day before oral administration, groups were separated into 10 groups, 5 for each group, and 5 for each group.

### 12-2. Administration of Test Substances

The test substance was prepared by dissolving it in physiological saline so that the dosage for experimental animals was 0, 750, 3,000, and 5,000 mg/kg, respectively, based on the content of the mutant yeast lysate KARC of the present invention.

The standards for administered dosage were in accordance with the Ministry of Food and Drug Safety's Korea national Toxicology Program (KNTP) toxicity test manual. The maximum application dose of 5,000 mg/kg guided by the KNTP manual was set as the maximum concentration for this experiment. The samples prepared for each group were orally administered once to each test animal. For the normal group (G1), physiological saline was administered.

### 12-3. Observation and Autopsy

For animals in all test groups, symptoms of mice were observed at least once a day from the date of acquisition to the date of necropsy. Symptoms were observed for 7 days after oral administration. After observing the rat's symptoms, an autopsy was performed. During the autopsy of the rat, changes in each organ were observed with the naked eye.

A single-dose toxicity test of the ALDH-containing KARC composition of the present invention was conducted using mice. As a result, no cases of mouse death were observed for 7 days at concentrations of the mutant yeast KARC up to 5,000 mg/kg. No unusual features, such as weight gain or changes in feed intake, were found in the mice. No unusual findings were found in the autopsy results conducted after the end of observation.

### [Example 13] Preparation of Food and Pharmaceutical Compositions for Preventing and Improving Fatty Liver Disease and Liver Dysfunction by Detoxifying Endogenous Acetaldehydes

Food and pharmaceutical compositions containing KARC as an active ingredient for prevention and recovery of fatty liver disease and liver dysfunction, were prepared. It is possible to prepare food or pharmaceutical compositions of various composition ratios containing KARC powder. As an example, the powder composition according to the present invention has the function of prevention of liver dysfunction and improvement of liver function through ingestion of 13 g of the composition twice a day. The weight ratio between the components and phases of the food or pharmaceutical composition containing the powder composition is shown in [Table 8].

TABLE 8

| | Ingredient | Ratio (wt %) |
|---|---|---|
| Food and pharmaceutical | KARC dry powder | 50 |
| compositions for prevention | Fructo-oligosaccharides | 9 |
| of liver dysfunction and | Stevia | 5 |
| improvement of liver | Citric acid anhydrous | 10 |
| function | Iso-malto | 4.3 |
| | Xylitol | 2.5 |
| | Citrus juice Powder | 6.2 |
| | Citrus Flavors Powder | 13 |

### INDUSTRIAL APPLICABILITY

In the food and pharmaceutical composition, KARC dry powder, excipients, and natural sweeteners such as fructo-oligosaccharides, enzyme-treated stevia (Stevia), anhydrous citric acid, iso-maltodextrins (Iso-malto), and xylitol, citrus juice powder, and citrus flavor powder were added. Processing and testing of raw materials and final products of food or pharmaceutical compositions were conducted in accordance with the general test methods and the Health Functional Foods Act described in the Korean Food Code.

KARC-containing foods or pharmaceutical compositions can prevent or improve liver function deterioration.

Through the above examples, the mutant yeast composition KARC containing aldehyde dehydrogenase was described in detail: therapeutically effective doses for disease model, manufacturing methods, pharmacological effects, administration methods, acute oral administration toxicity, and representative examples of food or pharmaceutical compositions. Although the efficacy of KARC has been described in detail through the above examples, these are only examples of the present invention.

A person skilled in the art can easily derive various modifications and other embodiments equivalent to the present invention from the above-described embodiments of the present invention.

Even foods or therapeutic agents containing a modified form of aldehyde dehydrogenase that embodies the technical gist of the present invention described in the patent claims fall within the scope of legal protection of the present invention.

1. A composition for alleviating endoplasmic reticulum stress, containing any one or a mixture thereof selected from the group consisting of Saccharomyces cerevisiae KCTC13925BP, KCTC14122BP, KCTC14123BP, KCTC14983BP, KCTC14984BP, and KCTC14985BP.

2. A food composition for inhibiting fatty liver disease containing any one or a mixture thereof selected from the group consisting of Saccharomyces cerevisiae KCTC13925BP, KCTC14122BP, KCTC14123BP, KCTC14983BP, KCTC14984BP, and KCTC14985BP.

3. A pharmaceutical composition for preventing or treating fatty liver symptoms containing any one or a mixture thereof selected from the group consisting of Saccharomyces cerevisiae KCTC13925BP, KCTC14122BP, KCTC14123BP, KCTC14983BP, KCTC14984BP, and KCTC14985BP.

4. A hepatitis prevention food composition containing any one or a mixture thereof selected from the group consisting of Saccharomyces cerevisiae KCTC13925BP, KCTC14122BP, KCTC14123BP, KCTC14983BP, KCTC14984BP, and KCTC14985BP.

5. A hepatitis treatment composition containing any one or a mixture thereof selected from the group consisting of Saccharomyces cerevisiae KCTC13925BP, KCTC14122BP, KCTC14123BP, KCTC14983BP, KCTC14984BP, and KCTC14985BP.
